(19) United States
(12) Patent Application Publication
(10) Pub. No.: US 2003/0003519 A1
(43) Pub. Date: Jan. 2, 2003

(54) Compositions and methods for the discovery and selection of biological information

(76) Inventor: Mehran M. Khodadoust, Brookline, MA (US)

Correspondence Address: CLARK & ELBING LLP, 101 FEDERAL STREET, BOSTON, MA, 02110, US

(21) Appl. No.: 10/029,471

(22) Filed: Oct. 25, 2001

Related U.S. Application Data
(63) Continuation-in-part of application No. 09/908,305, filed on Jul. 17, 2001.
Application No. 09/908,305 is a continuation-in-part of application No. 09/697,843, filed on Oct. 27, 2000, now abandoned.

(51) Int. Cl. G01N 33/567

(52) U.S. Cl. 435/7.21

(57) ABSTRACT

Methods and vectors are disclosed for discovering intracellular regulatory pathways utilized by specific stimulatory agents. Suitable stimulatory agents include cytokines, chemical agents, and antibodies. Cell lines and regulatory factors are provided for screening libraries of drug candidates to identify potential therapeutic agents. Methods and compositions are also provided for identifying genes which are necessary for, or capable of, up regulating or down regulating the targeted genomic loci in the selected cells.

### CROSS-REFERENCE TO RELATED APPLICATION

This application is a continuation-in-part of U.S. utility application Ser. No. 09/908,305, filed Jul. 17, 2001, which is still pending and is a continuation-in-part of U.S. utility application Ser. No. 09/697843, filed Oct. 27, 2000.

### BACKGROUND OF THE INVENTION

The present invention relates to compositions and methods which can be used to obtain biological information from cells. Such information, which includes the regulatory pathways and components utilized by ligands and cytokines to regulate the expression of genes in a variety of specific cells and tissue types, can be used to maximize a drug targeting strategy for the selection of drug candidates and for library screening. Vectors are modified to introduce regulatory or reporter genes into regulated loci within the genome of a desired cell line. Such vectors utilize positive and negative selection markers for the identification and delivery of genes to the regulated loci. Individual isolated cell clones that express the reporter marker in a stimulation-dependent manner are used as reporter cell lines to test the functional activity of the ligand in the cells. The cell clones can be used to identify key regulatory factors, such as promoters and enhancers that are dependent on stimulation by the ligand and genes regulated by the ligand. Genes that can control the response of a regulated loci to stimulation by a ligand or cytokine in the selected cell or tissue type can also be identified.

Modern drug discovery techniques are increasingly based on genomics and depend upon the identification of specific genomic targets and regulatory pathways. These genomic targets include the specific genes of interest and their cell-specific regulatory control elements, such as enhancers and promoters. The expression of regulatory factors occurs in a variety of specific cell and tissue types and involves a multiplicity of pathways in an organism. An inhibitor or antagonist for a given factor can have unintended consequences if these complexities are not fully explored and resolved. See, for instance, Khodadoust et al., Blood, 92, No. 7, pages 2399-2409 (1998), which describes the distinct regulatory mechanisms for IFN-α/β and IFN-γ-mediated induction of the Ly-6E gene in B cells. Through deletion analysis, it was found that a cooperative interaction exists between physically disparate regulatory regions of the gene. This indicates the complexity involved in achieving cell-type specificity in IFN-mediated gene regulation and is an example of the complexity involved in dealing with these effects.

Regulation of gene expression can be investigated by the integration of promoterless selectable marker genes into the chromosomal loci of cells and the subsequent identification of the active loci. This type of “induction trap”strategy has been used to identify specific enhancers, promoters, and other regulatory elements of genes of interest. Induction gene trap vectors, which generate spliced fusion transcripts between the reporter gene and the endogenous gene present at the site of integration, are used to identify regulated gene loci. This approach can be used to distinguish between genes involved in specific regulatory pathways and the “housekeeping” genes, which are turned on independently of activation by a ligand. The genes regulated by a ligand would be implicated in regulatory pathways of interest that can be harnessed in drug development.

Gene trap vectors, which generally consist of a splice-acceptor site located upstream from a reporter gene, target the introns of the eukaryotic genome. Integration of the reporter into an intron results in a fusion transcript containing mRNA from the endogenous gene and from the reporter gene sequence. The use of an IRES site between the splice acceptor and the reporter gene of a gene trap vector means that the reporter gene product and the endogenous gene product need not be fusion products, thereby increasing the likelihood that integration of the vector will result in expression of the reporter gene product. Gene entrapment vectors, or gene trap vectors, are tools which are frequently used for gene discovery and elucidation. These vectors can be employed to identify developmentally regulated genes.

U.S. Pat. No. 5,922,601 describes an induction gene trap construct used for the identification of genes that are regulated upon the occurrence of a cellular transition event. The construct contains a functional splice acceptor, a translation stop sequence, an internal ribosome entry site (“IRES”), and a promoterless protein coding sequence encoding a polypeptide providing positive and negative selection traits. The positive and negative selection traits can be introduced by employing nucleic acid encoding a single protein whose expression (or non-expression) can be detected as a positive or negative selection trait. Typical proteins of this type include neomycin phosphotransferase and thymidine kinase. The construct is incorporated in a vector which is introduced into a cell, and the expression of the positive and negative selection traits before and after occurrence of the transition event is detected by means of drug selection. The transition event is typically the transition from an undifferentiated cell to a differentiated cell. The gene trap vector of this reference allows for the selection of genes at the cell populations in which a trapped locus is either active or becomes inactive as a result of a cellular transition event.

Mainguy et al., Nature Biotechnology, 18, pages 746-749 (2000) describes vectors for use as induction gene traps to identify homeoprotein target genes. The vectors used in this reference include the PT2 bicistronic gene containing the lacZ gene fused to a splice acceptor, a thymidine kinase gene driven by an IRES, and a Neo gene under the control of the phosphoglycerate kinase promoter. The PT2 gene trap vector allows the use of gancyclovir for selecting against integration of the vector into constitutively active genes. Hence, subsequent activation allows for the selection and isolation of regulated genes. Using this vector, an embryonic stem cell gene trap library was constructed and screened for activity towards engrailed homeodomain protein. See, also, European Patent No. 902,092, which discloses a similar procedure.

U.S. Pat. No. 5,928,888 describes an induction gene trap method, identifying active genomic polynucleotides for identifying proteins and compounds that modulate genomic polynucleotides. The reference achieves this result by inserting a beta-lactamase polynucleotide into the genome of a eukaryotic cell. The cell is then contacted with a predetermined amount of an agent which activates the beta-lactamase, and the amount of beta-lactamase activity is measured. The expressing and non-expressing cells are separated, and the integration of the beta-lactamase gene in the genome of the cells is determined. The reference states that the beta-lactamase reporter provides a mechanism for preparing a genomic integration assay for drug discovery in a high throughput format.

See, also, Whitney et al., Nature Biotechnology, 16, pages 1329-1333 (1998), which describes a genome-wide functional assay for the rapid isolation of cell clones and genetic elements responsive to specific stimuli. This assay uses a promoterless beta-lactamase reporter gene transfected into a human T-cell line to generate a living library of reporter-tagged clones. Flow cytometry and fluorogenic substrates were used to identify patterns of regulation associated with specific genes.

PCT published application, publication number WO 99/02719, discloses methods and DNA constructs which can be used for the detection and manipulation of a target eukaryotic gene whose expression is restricted to specific tissue or specialized cell types. According to this reference, an embryonic stem cell is transformed with a vector containing a first component under the control of a promoter which has restricted expression in a particular cell or tissue type. The stem cell is also transformed with a gene trap vector encoding a second indicator component. The indicators act in a complementary way to produce a detectable signal, such as the omega and alpha components of β-galactosidase which combine to form the complete enzyme. Measurement of the detectable enzyme indicates that the gene of the gene trap vector has been integrated into the genome of the selected cell type.

The activity of a ligand in an organism involves a multiplicity of regulatory pathways depending on the specific cell or tissue type under investigation. For instance, a particular growth factor, such as stem cell factor (“SCF”), can activate cells unrelated to the cell type under investigation, such as mast cells. This lack of specificity, redundancy, and potential toxicity creates a complication when using these factors as protein therapeutics or when developing, for instance, inhibitors, antagonists, or agonists to these factors, since these factors, inhibitors, or antagonists may act on the unrelated cells in unfavorable ways.

It is therefore an objective of this invention to provide a method for characterizing the response of an organism to a ligand by identifying the genes and regulatory mechanisms, in specific cells and tissues, which are activated by the ligand. It is also an object of this invention to discover genes, cells containing the genes and gene regulatory mechanisms which can be used to screen libraries of compounds to find potential drug candidates of interest.

### SUMMARY OF THE INVENTION

The present invention relates broadly to the identification of cellular pathways utilized by ligands of interest to regulate specific genomic loci, and to polynucleotide segments which can be incorporated into induction gene trap vectors in a manner such that they would be operably linked to the regulated loci in the specific transfected eukaryotic cells. The use of the incorporated polynucleotides, or the proteins coded by the polynucleotides, for the selection and identification of cells which are responsive to one or more stimulatory agents, provides a panel of cell clones which can be used to dissect regulatory mechanisms. These polynucleotides and proteins can be used to screen a library of drug candidates to obtain promising therapeutic agents for farther evaluation. In addition, the protein coded by the polynucleotide can be used as a means to influence the expression of a gene trap vector. This allows for the isolation and identification of the genes, which affect the up or down regulation of the genomic loci in such cells. These genes may be used to directly screen libraries for therapeutic agents. The genes themselves may have therapeutic applications in, for example, combination therapies or drug discovery.

The current use of protein therapeutics, such as ligands, cytokines or antibodies, and approaches for selecting antagonists, inhibitory agents, and agonists against these factors for use in drug therapy, fail to take into account ligand redundancies and the selectivity of specific cell and tissue types with respect to regulatory pathways under investigation. Therapeutics selected in this way can have unintended consequences, such as toxicity and the selection of inhibitory agents which have an adverse impact on the regulation of cells and tissue not implicated in the disease. Additional complexity is introduced when it is desired to use more than one ligand in combination therapies to achieve a desired therapeutic effect. The use of multiple ligands can involve numerous regulatory pathways in divergent cell types, and this can also have unintended consequences for the regulation of cells which are implicated in the disease under investigation. However, there are currently no simple solutions to this problem.

The vectors and methods of this invention can be used to generate a panel or library of cells under the control of regulatory elements for one or more stimulatory agents of interest. This collection of cells can be used, in turn, as targets to screen libraries of drug candidates to identify potential therapeutics having a high level of specificity, safety, and effectiveness. In addition, genes which are under the control of the regulatory elements and are responsive to specific stimulatory agents of interest, and genes which regulate the genomic loci and effect the expression of such loci, can be identified, isolated and characterized. These genes, or their regulatory factors, can also be used to screen libraries of drug candidates in a variety of assay formats, such as cell based assays. Stimulatory agents of interest include insulin, stem cell factor (“SCF”), vascular endothelial growth factor (“VEGF”), IL-2, IL-3, IL-6, IgE, FGF-1, FGF-2, FGF-3, TGF-β, TNF-β, and TNF-α.

Accordingly, in one aspect, the present invention includes nucleic acid constructs and vectors containing the constructs, for infecting cells and for generating a panel of cell clones which can be used as screening tools to screen libraries of candidate drug molecules.

According to this aspect, in one embodiment a nucleic acid construct for use in preparing a vector for generating a panel of cells comprises the following elements in downstream (5′ to 3′) sequence: a cassette containing an internal ribosome entry site; a transactivator polypeptide coding sequence encoding a polypeptide, said polypeptide acting as a regulator unit to one or more regulatory elements contained in a genomic loci in a particular cell or cell type of interest, said transactivator polypeptide being responsive to one or more regulatory elements contained in a genomic loci in a cell of interest; a translation stop sequence; an internal ribosome entry site; a reporter element responsive to at least one stimulatory agent; and a translation stop sequence.

In preferred features of this embodiment of the invention, the reporter element can be an enzyme, such as secreted alkaline phosphatase, Luciferase™, or green fluorescent protein (“GFP”), the marker polypeptide can be a the promoterless protein coding sequence, such as a tetracycline regulator unit (tTA). A nucleic acid cassette containing these elements can be incorporated into an induction gene trap vector containing a splice acceptor site; an internal ribosome entry site; a marker polypeptide coding sequence encoding a polypeptide providing selection traits and being responsive to one or more regulatory elements contained in a genomic loci in a particular cell or cell type of interest; and a translation stop sequence. The marker polypeptide can be a fusion protein with positive and negative selection traits. Negative selection traits can be provided in situations whereby the expressed gene leads to the elimination of the host cell, frequently in the presence of a nucleoside analog, such as gancyclovir. Positive selection traits can be provided by drug resistance genes. Suitable negative selection markers include, for example, DNA sequences encoding Hprt, gpt, HSV-tk, diphtheria toxin, ricin toxin, and cytosine deeaminase. Suitable positive selection traits include, for example, DNA sequences encoding neomycin resistance, hygromycin resistance, histidinol resistance, xanthine utilization, Zeocin resistance, and bleomycin resistance. A particularly preferred fusion protein is a fusion protein encoding Tk-Zeo.

This nucleic acid construct can be incorporated into a vector, such as a viral vector, and preferably a retroviral vector, to transfect cells of interest. This can be accomplished by introducing the vector into a medium containing the cells using techniques known to those skilled in the art. Suitable techniques are described in U.S. Pat. No. 5,922,601, the disclosure of which is incorporated herein by reference in its entirety. Not all of the cells will be successfully transfected, meaning that the vector will not be integrated into the genomic loci of the cell. Successful integration events can be selected for using a drug selection compound, such as zeocin. If the vector contains a zeocin resistant gene, the zeocin will serve to kill the cells in which the vector has not been successfully integrated into the genome of the cell.

Once cells which have been successfully transfected, and the vector has been operably integrated into the genome of the cell, the cells are selected for activity with respect to specific stimulatory agents. Such activity can include those cells in which regulatory factors, such as enhancers and promoters, have been turned on by the stimulatory agent, and those cells in which the appropriate regulatory factors have been turned off by the stimulatory agent. Each of these cases will involve a different selection protocol.

To select for cells which have been turned on by the stimulatory agent, the stimulatory agent is introduced into the culture medium with zeocin, a positive selective agent, or another appropriate drug selection agent. The stimulatory agent can be added to the medium either prior to, subsequent to, or together with the drug selection agent. The stimulatory agent activates the promoterless first marker polypeptide coding sequence in the vector, which encodes a polypeptide conferring drug resistance to the drug selection agent. Regulatory factors present in housekeeping genes present in the cell loci can also be activated independently of the stimulatory agent. However, housekeeping genes are not specific for the stimulatory agent and must be eliminated. This is essential in order to obtain isolated cultures of cells specific for the stimulatory agent. This can be accomplished by adding a negative selection agent such as gancyclovir, for example, which is acted on by the TK (thymidine kinase) gene to eliminate cells expressing the protein. Those cells remaining in the cell culture after treatment with gancyclovir are clones with the vector inserted into the genomic loci which are turned on by the particular stimulatory agent. The expression of the reporter (SEAP) gene, which is turned on by the stimulatory agent, can be used as the readout, allowing the cells to be used directly as drug targets to screen libraries of compounds, or treated with other stimulatory agents in the same manner indicated above in order to identify clones which are capable of being turned on by more than one stimulatory agent.

To select for cells which have been turned off by the stimulatory agent, Zeocin is first introduced into the cell culture medium to eliminate those cells which do not have the vector integrated into the genome of the cell. The stimulatory agent and gancyclovir are then added to a medium containing the cells, and the housekeeping genes which are active in the cell are eliminated. However, the cell clones which are turned off upon treatment with the stimulatory agent remain in the culture. These cells can also be used as drug targets, or treated with other stimulatory agents as indicated above for the selection of genes which are turned on by the stimulatory agent. As will be appreciated, other possible combinations for cell selection using several stimulatory agents can be readily envisioned.

In another embodiment, the nucleic acid construct can contain a promoter regulating the expression of the marker polypeptide coding sequence. The promoter acts on one of the polynucleotide sequences which comprise the marker polypeptide encoding region of the construct. Preferably, the polynucleotide sequence encodes a positive selection marker, such as Zeocin. In this embodiment, the promoter is phosphoglycerate kinase (“PGK”).

This vector can also be used to generate a panel or library of cells under the control of regulatory elements activated by one or more stimulatory agents of interest using the method set forth below.

To select for cells which have been turned on by the stimulatory agent, the cells are transformed with the vector containing a promoter for the selection marker. A selection drug, such as zeocin, is introduced into the culture medium to eliminate cells in which the vector has not been integrated into the genomic loci. The culture medium is changed, and gancyclovir is introduced to eliminate housekeeping genes which are active and express Tk. A stimulatory agent is then added to the medium, and the amount of secreted alkaline phosphatase is measured as a positive indicator of the presence of cells which are responsive to the stimulatory agent. Those cells generating SEAP and which are turned on by the stimulatory agent are selected, separated, and used as drug targets.

To select for cells which have been turned off by the stimulatory agent, zeocin is introduced into the cell culture medium to eliminate those cells which do not have the vector integrated into the genome of the cell. The stimulatory agent and gancyclovir are then added to a medium containing the cells, and the housekeeping genes which are active in the cell are eliminated. The medium is changed, and the cell clones which produce secreted alkaline phosphatase in the absence of the stimulatory agent are selected by measuring the amount of secreted alkaline phosphatase produced. These cells can also be used as drug targets, or treated with other stimulatory agents to prepare cells which are specific for more than one stimulatory agent.

In another aspect, a method is provided for selecting cells and cell clones from a medium containing a collection of cells having a specific response to a selected stimulatory agent. The method involves transforming eukaryotic cells with an induction gene trap vector to operably integrate the vector into the genome of the cell. The vector can be any vector, such as the vectors described previously, which includes a marker polypeptide coding sequence and a reporter element. Optionally, the vector can also include a transactivator coding sequence. Cells are selected having one or more regulatory elements which activate the reporter polypeptide and, if present, the transactivator. Cells which are specifically activated by the stimulatory agent are selected. These cells can then be used in cell-based assays for screening libraries of potential therapeutic agents.

In yet another aspect, a method is provided for selecting cells and cell clones from a medium containing a collection of cells having a specific response to a selected stimulatory agent. The method involves transforming eukaryotic cells with an induction gene trap vector to operably integrate the vector into the genome of the cell. The vector can be any vector, such as the vectors described previously, which includes a marker polypeptide coding sequence and a reporter element. Optionally, the vector can also include a transactivator coding sequence. Cells are selected having one or more regulatory elements which activate the reporter gene and, if present, the transactivator. Cells which are specifically inactivated by the stimulatory agent are selected. These cells can then be used in cell-based assays for screening libraries of potential therapeutic agents.

In a further aspect of the invention, a gene trap vector can be used to identify genes that lead to transcriptional control, or which up regulate or down regulate the genomic loci of the isolated cell clones. These vectors can be viral vectors, and preferably retroviral vectors, which are integrated into the genomic loci of the cell.

This vector contains a nucleic acid construct including, in 5′ to 3′ sequence: a minimal promoter sequence containing a transactivator regulatory element, such as a tetracycline responsive element; a protein coding sequence encoding a marker polypeptide providing positive selection traits, the protein being responsive to the transactivator regulatory elements; an internal ribosome entry site; and a functional splice donor site. The tetracycline regulator unit introduced by the induction gene trap vector generates a protein which is activated or repressed by tetracycline in an “on/off” mode and binds to the tetracycline responsive element within the minimal promoter sequence of the gene trap vector and leads to its transcriptional control.

When the cell is contacted with a stimulatory agent, the tetracycline regulator protein is turned on. This protein binds to the minimal promoter sequence containing the tetracycline responsive element, causing the promoter (“TRE_{Pcmv}”) to activate and cause transcription of a gene downstream of the promoter. This gene, in turn, up regulates or down regulates the genomic loci, causing the tetracycline regulator unit to express protein, thereby activating the TRE_{Pcmv} promoter/transactivator to transcribe additional copies of the gene, and so on. Eventually, as a result of this feedback process, enough genetic material will be generated to be detected and identified.

The net result of this process is to isolate genes that up regulate or down regulate the loci of cells which respond to stimulation by one or more stimulatory agents. These genes can then be used as drug targets or as potential therapeutics (e.g., as gene therapy constructs or antisense molecules). Regulatory elements contained in the gene, such as promoters and enhancers, can also be isolated, characterized, and used for drug discovery.

The use of the cells, genes, and regulatory elements of this invention to select drug candidates for use as therapeutic agents is conventional in the art. For instance, the cells can be used in live cell screening assays which are effective to evaluate the specificity, toxicity and dosage of a selected therapeutic agent. If live cell assays are not available, conventional assay screening techniques can be used.

In another aspect, the invention provides a method of selecting for one or more cells having a specific response to a stimulatory agent of interest. This method involves inserting a vector including a cassette having a positive selection marker, a negative selection marker, and a reporter gene into eukaryotic cells under conditions that result in the integration of the cassette into the genome of the cells. The reporter gene is operably linked to an endogenous regulatory element in at least one cell. Cells in which expression of the reporter gene is specifically activated by the stimulatory agent are selected. In particular embodiments, this selection step may involve incubating the cells in the presence of the stimulatory agent and a positive selection agent and incubating the cells under conditions in which a negative selection agent is present and the stimulatory agent is absent. In other embodiments, the selection step involves incubating the cells in the presence of a positive selection agent, incubating the cells in the presence of a negative selection agent, incubating the cells in the presence of the stimulatory agent, and selecting the cells that express the reporter gene in the presence of the stimulatory agent. In yet other embodiments, the vector does not contain a promoter operably linked to the reporter gene. In various embodiments, the vector further includes a nucleic acid segment encoding a transactivator polypeptide (e.g., tTA) that is integrated into the genome of the cells. The nucleic acid segment encoding a transactivator polypeptide may be operably linked to a promoter in the vector or may not be operably linked to a promoter in the vector. Desirably, the nucleic acid encoding the transactivator polypeptide is integrated into the genome of the cells under the control of an endogenous regulatory element. Optionally, the methods may include identifying the regulatory element that is activated by the stimulatory agent. In various embodiments, the positive selection marker is operably linked to a prokaryotic promoter in the cassette that integrates into the genome of the eukaryotic cells. In this case, the regulatory element activated by the stimulatory agent may be identified by (i) inserting a nucleic acid that includes the positive selection marker and a segment of the eukaryotic genome flanking the integrated cassette into bacterial cells under conditions that allow the selection of bacterial cells expressing the positive selection marker under the control of the prokaryotic promoter, (ii) amplifying the segment of the eukaryotic genome that is inserted into the selected bacterial cells, and (iii) sequencing the amplified segment. In other embodiments, the positive selection marker is operably linked to a yeast promoter in the cassette that integrates into the genome of the eukaryotic cells. In this case, the regulatory element activated by the stimulatory agent may be identified by (i) inserting a nucleic acid that includes the positive selection marker and a segment of the eukaryotic genome flanking the integrated cassette into yeast cells under conditions that allow the selection of yeast cells expressing the positive selection marker under the control of the yeast promoter, (ii) amplifying the segment of the eukaryotic genome that is inserted into the selected yeast cells, and (iii) sequencing the amplified segment.

In a related aspect, the invention provides another method of selecting for one or more cells having a specific response to a stimulatory agent of interest. This method involves inserting a vector including a cassette having a positive selection marker, a negative selection marker, and a nucleic acid segment encoding a transactivator polypeptide into eukaryotic cells under conditions that result in the integration of the cassette into the genome of the cells. The transactivator polypeptide is operably linked to an endogenous regulatory element in at least one cell. Cells in which expression of the transactivator polypeptide is specifically activated by the stimulatory agent are selected. In particular embodiments, this selection step may involve incubating the cells in the presence of the stimulatory agent and a positive selection agent and incubating the cells under conditions in which a negative selection agent is present and the stimulatory agent is absent. In other embodiments, the selection step involves incubating the cells in the presence of a positive selection agent, incubating the cells in the presence of a negative selection agent, incubating the cells in the presence of the stimulatory agent, and selecting the cells that express the transactivator polypeptide in the presence of the stimulatory agent. In yet other embodiments, the vector does not contain a promoter operably linked to the nucleic acid segment encoding a transactivator polypeptide. In various embodiments, the vector further includes a reporter gene that is integrated into the genome of the cells. The reporter gene may be operably linked to a promoter in the vector or may not be operably linked to a promoter in the vector. Desirably, the reporter gene is integrated into the genome of the cells under the control of an endogenous regulatory element. The methods may optionally include identifying the regulatory element that is activated by the stimulatory agent. In various embodiments, the positive selection marker is operably linked to a prokaryotic promoter in the cassette that integrates into the genome of the eukaryotic cells. In this case, the regulatory element activated by the stimulatory agent may be identified by (i) inserting a nucleic acid that includes the positive selection marker and a segment of the eukaryotic genome flanking the integrated cassette into bacterial cells under conditions that allow the selection of bacterial cells expressing the positive selection marker under the control of the prokaryotic promoter, (ii) amplifying the segment of the eukaryotic genome that is inserted into the selected bacterial cells, and (iii) sequencing the amplified segment. In other embodiments, the positive selection marker is operably linked to a yeast promoter in the cassette that integrates into the genome of the eukaryotic cells. In this case, the regulatory element activated by the stimulatory agent may be identified by (i) inserting a nucleic acid that includes the positive selection marker and a segment of the eukaryotic genome flanking the integrated cassette into yeast cells under conditions that allow the selection of yeast cells expressing the positive selection marker under the control of the yeast promoter, (ii) amplifying the segment of the eukaryotic genome that is inserted into the selected yeast cells, and (iii) sequencing the amplified segment.

In a related aspect, the invention provides yet another method of selecting for one or more cells having a specific response to a stimulatory agent of interest. This method includes inserting a vector including a cassette having a positive selection marker, a negative selection marker, and a reporter gene into eukaryotic cells under conditions that result in integration of the cassette into the genome of the cells. The reporter gene is operably linked to an endogenous regulatory element in at least one cell. Cells in which expression of the reporter gene is specifically inactivated by the stimulatory agent are selected. In various embodiments, this selection involves incubating the cells in the presence of a positive selection agent and incubating the cells in the presence of the stimulatory agent and a negative selection agent. In yet other embodiments, the vector does not contain a promoter operably linked to the reporter gene. In various embodiments, the vector further includes a nucleic acid segment encoding a transactivator polypeptide (e.g., tTA) that is integrated into the genome of the cells. The nucleic acid segment encoding a transactivator polypeptide may be operably linked to a promoter in the vector or may not be operably linked to a promoter in the vector. Desirably, the nucleic acid encoding the transactivator polypeptide is integrated into the genome of the cells under the control of an endogenous regulatory element. In other embodiments, the method also includes identifying the regulatory element that is inactivated by the stimulatory agent. In various embodiments, the positive selection marker is operably linked to a prokaryotic promoter in the cassette that integrates into the genome of the eukaryotic cells. In this case, the regulatory element inactivated by the stimulatory agent may be identified by (i) inserting a nucleic acid that includes the positive selection marker and a segment of the eukaryotic genome flanking the integrated cassette into bacterial cells under conditions that allow the selection of bacterial cells expressing the positive selection marker under the control of the prokaryotic promoter, (ii) amplifying the segment of the eukaryotic genome that is inserted into the selected bacterial cells, and (iii) sequencing the amplified segment. In other embodiments, the positive selection marker is operably linked to a yeast promoter in the cassette that integrates into the genome of the eukaryotic cells. In this case, the regulatory element inactivated by the stimulatory agent may be identified by (i) inserting a nucleic acid that includes the positive selection marker and a segment of the eukaryotic genome flanking the integrated cassette into yeast cells under conditions that allow the selection of yeast cells expressing the positive selection marker under the control of the yeast promoter, (ii) amplifying the segment of the eukaryotic genome that is inserted into the selected yeast cells, and (iii) sequencing the amplified segment.

In a related aspect, the invention provides still another method of selecting for one or more cells having a specific response to a stimulatory agent of interest. This method includes inserting a vector including a cassette having a positive selection marker, a negative selection marker, and a nucleic acid segment encoding a transactivator polypeptide into eukaryotic cells under conditions that result in integration of the cassette into the genome of the cells. The nucleic acid segment encoding a transactivator polypeptide is operably linked to an endogenous regulatory element in at least one cell. Cells in which expression of the transactivator polypeptide is specifically inactivated by the stimulatory agent are selected. In various embodiments, this selection involves incubating the cells in the presence of a positive selection agent and incubating the cells in the presence of the stimulatory agent and a negative selection agent. In yet other embodiments, the vector does not contain a promoter operably linked to the nucleic acid segment encoding a transactivator polypeptide. In various embodiments, the vector further includes a reporter gene that is integrated into the genome of the cells. The reporter gene may be operably linked to a promoter in the vector or may not be operably linked to a promoter in the vector. Desirably, the reporter gene is integrated into the genome of the cells under the control of an endogenous regulatory element. The method may optionally include identifying the regulatory element that is inactivated by the stimulatory agent. In various embodiments, the positive selection marker is operably linked to a prokaryotic promoter in the cassette that integrates into the genome of the eukaryotic cells. In this case, the regulatory element inactivated by the stimulatory agent may be identified by (i) inserting a nucleic acid that includes the positive selection marker and a segment of the eukaryotic genome flanking the integrated cassette into bacterial cells under conditions that allow the selection of bacterial cells expressing the positive selection marker under the control of the prokaryotic promoter, (ii) amplifying the segment of the eukaryotic genome that is inserted into the selected bacterial cells, and (iii) sequencing the amplified segment. In other embodiments, the positive selection marker is operably linked to a yeast promoter in the cassette that integrates into the genome of the eukaryotic cells. In this case, the regulatory element inactivated by the stimulatory agent may be identified by (i) inserting a nucleic acid that includes the positive selection marker and a segment of the eukaryotic genome flanking the integrated cassette into yeast cells under conditions that allow the selection of yeast cells expressing the positive selection marker under the control of the yeast promoter, (ii) amplifying the segment of the eukaryotic genome that is inserted into the selected yeast cells, and (iii) sequencing the amplified segment.

In another aspect, the invention provides a method for identifying a nucleic acid of interest that encodes a protein that modulates the activity of a regulatory element in a cell. This method includes inserting a first vector including a first cassette having a positive selection marker, a negative selection marker, a reporter gene, and a nucleic acid segment encoding a transactivator polypeptide into eukaryotic cells under conditions that result in integration of the first cassette into the genome of the cells. The reporter gene is operably linked to an endogenous regulatory element in at least one cell, or the reporter gene is operably linked to a regulatory element in the first vector. A second vector including a second cassette having a promoter operably linked to a responsive element that is responsive to the transactivator polypeptide is also inserted into the cells under conditions that result in integration of the second cassette into the genome of the cells. The promoter is operably linked to an endogenous nucleic acid of interest encoding a protein that modulates (i.e., increases or decreases) the activity of the regulatory element in at least one cell. Cells that have an altered level of reporter gene expression under conditions that activate the transactivator polypeptide are selected. Desirably, the nucleic acid of interest from at least one selected cell is identified. The nucleic acid of interest is determined to encode a protein that activates the regulatory element if the cells have increased reporter gene expression under conditions that activate the transactivator polypeptide. Or the nucleic acid of interest is determined to encode a protein that inactivates the regulatory element if the cells have decreased reporter gene expression under conditions that activate the transactivator polypeptide. In desirable embodiments, the transactivator polypeptide is tTA, and the responsive element comprises a tetracycline responsive element. In various embodiments, the first vector contains a regulatory element that was identified as being regulated by a stimulatory agent of interest. For example, the methods of the invention may be used to identify an endogenous regulatory element that is regulated by a stimulatory agent, and then this regulatory element may be cloned into the first vector using standard methods and used to identify endogenous nucleic acids encoding proteins that modulate the regulatory element. In other embodiments, the second vector further includes a positive selection marker which is integrated into the genome of the cells. In still other embodiments, the positive selection marker in the second vector (denoted the second positive selection marker) is operably linked to a prokaryotic promoter in the second cassette that integrates into the genome of the eukaryotic cells. In this case, the nucleic acid encoding a protein that modulates the activity of the regulatory element may be identified by (i) inserting a nucleic acid that includes the second positive selection marker and a segment of the eukaryotic genome flanking the second integrated cassette into bacterial cells under conditions that allow the selection of bacterial cells expressing the second positive selection marker under the control of the prokaryotic promoter, (ii) amplifying the segment of the eukaryotic genome that is inserted into the selected bacterial cells, and (iii) sequencing the amplified segment. In yet other embodiments, the positive selection marker in the second vector is operably linked to a yeast promoter in the second cassette that integrates into the genome of the eukaryotic cells. In this case, the nucleic acid encoding a protein that modulates the activity of the regulatory element may be identified by (i) inserting a nucleic acid that includes the second positive selection marker and a segment of the eukaryotic genome flanking the second integrated cassette into yeast cells under conditions that allow the selection of yeast cells expressing the second positive selection marker under the control of the yeast promoter, (ii) amplifying the segment of the eukaryotic genome that is inserted into the selected yeast cells, and (iii) sequencing the amplified segment.

In a related aspect, the invention features another method for identifying a nucleic acid of interest that encodes a protein that modulates the activity of a regulatory element in a cell. This method involves inserting a first vector including a first cassette having a positive selection marker, a negative selection marker, and a recombinase signal sequence into eukaryotic cell under conditions that result in the integration of the first cassette into the genome of the cells. A second vector including a second cassette that includes a recombinase signal sequence, a nucleic acid segment encoding a transactivator polypeptide, and a reporter gene is inserted into the cells under conditions that result in recombination between the recombinase signal sequence in the second vector and the recombinase signal sequence integrated into the genome of the cells. This recombination results in the integration of the second cassette into the genome of the cells such that the reporter gene is operably linked to a regulatory element in at least one cell. The regulatory element may be an endogenous regulatory element, or the regulatory element may be a regulatory element of interest from the first or second vector. A third vector including a third cassette having a promoter operably linked to a responsive element that is responsive to the transactivator polypeptide is inserted into the cells. This step results in integration of the third cassette into the genome of the cells such that the promoter is operably linked to an endogenous nucleic acid of interest encoding a protein that modulates (i.e., increases or decreases) the activity of the regulatory element in at least one cell. The cells that have an altered level of reporter gene expression under conditions that activate the transactivator polypeptide are selected. Desirably, the nucleic acid of interest is identified from at least one selected cell. The nucleic acid of interest is determined to encode a protein that activates the regulatory element if the cells have increased reporter gene expression under conditions that activate the transactivator polypeptide. Alternatively, the nucleic acid of interest is determined to encode a protein that inactivates the regulatory element if the cells have decreased reporter gene expression under conditions that activate the transactivator polypeptide. Desirably, the transactivator polypeptide is tTA, and the responsive element comprises a tetracycline responsive element. Exemplary recombinase signal sequences include LoxP sites, Lox 511 sites, and any other recombinase signal sequence described herein. In various embodiments, the first and/or second vector include two recombinase signal sequences, such as two LoxP sites. In various embodiments, the first vector, second vector, third vector, or another vector inserted into the cells encodes a recombinase that recognizes the recombinase signal sequence. In desirable embodiments, the recombinase signal sequence(s) in the first vector are identical to those in the second vector. In various embodiments, the first or second vector contains a regulatory element that was identified as being regulated by a stimulatory agent of interest. For example, the methods of the invention may be used to identify an endogenous regulatory element that is regulated by a stimulatory agent, and then this regulatory element may be cloned into the first or second vector using standard methods and used to identify endogenous nucleic acids encoding proteins that modulate the regulatory element. In other embodiments, the third vector further includes a positive selection marker which is integrated into the genome of the cells. In still other embodiments, the positive selection marker in the third vector (denoted the second positive selection marker) is operably linked to a prokaryotic promoter in the third cassette that integrates into the genome of the eukaryotic cells. In this case, the nucleic acid encoding a protein that modulates the activity of the regulatory element may be identified by (i) inserting a nucleic acid that includes the second positive selection marker and a segment of the eukaryotic genome flanking the third integrated cassette into bacterial cells under conditions that allow the selection of bacterial cells expressing the second positive selection marker under the control of the prokaryotic promoter, (ii) amplifying the segment of the eukaryotic genome that is inserted into the selected bacterial cells, and (iii) sequencing the amplified segment. In yet other embodiments, the positive selection marker in the third vector is operably linked to a yeast promoter in the third cassette that integrates into the genome of the eukaryotic cells. In this case, the nucleic acid encoding a protein that modulates the activity of the regulatory element may be identified by (i) inserting a nucleic acid that includes the second positive selection marker and a segment of the eukaryotic genome flanking the third integrated cassette into yeast cells under conditions that allow the selection of yeast cells expressing the second positive selection marker under the control of the yeast promoter, (ii) amplifying the segment of the eukaryotic genome that is inserted into the selected yeast cells, and (iii) sequencing the amplified segment.

In a related aspect, the invention features yet another method for identifying a nucleic acid of interest that encodes a protein that modulates the activity of a regulatory element in a cell. This method involves inserting a first vector including a first cassette having a positive selection marker, a negative selection marker, a reporter gene, and a recombinase signal sequence into eukaryotic cell under conditions that result in the integration of the first cassette into the genome of the cells. A second vector including a second cassette that includes a recombinase signal sequence, and a nucleic acid segment encoding a transactivator polypeptide, is inserted into the cells under conditions that result in recombination between the recombinase signal sequence in the second vector and the recombinase signal sequence integrated into the genome of the cells. This recombination results in the integration of the second cassette into the genome of the cells such that the reporter gene is operably linked to a regulatory element in at least one cell. The regulatory element may be an endogenous regulatory element, or the regulatory element may be a regulatory element of interest from the first or second vector. A third vector including a third cassette having a promoter operably linked to a responsive element that is responsive to the transactivator polypeptide is inserted into the cells. This step results in integration of the third cassette into the genome of the cells such that the promoter is operably linked to an endogenous nucleic acid of interest encoding a protein that modulates (i.e., increases or decreases) the activity of the regulatory element in at least one cell. The cells that have an altered level of reporter gene expression under conditions that activate the transactivator polypeptide are selected. Desirably, the nucleic acid of interest is identified from at least one selected cell. The nucleic acid of interest is determined to encode a protein that activates the regulatory element if the cells have increased reporter gene expression under conditions that activate the transactivator polypeptide. Alternatively, the nucleic acid of interest is determined to encode a protein that inactivates the regulatory element if the cells have decreased reporter gene expression under conditions that activate the transactivator polypeptide. Desirably, the transactivator polypeptide is tTA, and the responsive element comprises a tetracycline responsive element. Exemplary recombinase signal sequences include LoxP sites, Lox 511 sites, and any other recombinase signal sequence described herein. In various embodiments, the first and/or second vector include two recombinase signal sequences, such as two LoxP sites. In various embodiments, the first vector, second vector, third vector, or another vector inserted into the cells encodes a recombinase that recognizes the recombinase signal sequence. In desirable embodiments, the recombinase signal sequence(s) in the first vector are identical to those in the second vector. In various embodiments, the first or second vector contains a regulatory element that was identified as being regulated by a stimulatory agent of interest. For example, the methods of the invention may be used to identify an endogenous regulatory element that is regulated by a stimulatory agent, and then this regulatory element may be cloned into the first or second vector using standard methods and used to identify endogenous nucleic acids encoding proteins that modulate the regulatory element. In other embodiments, both the first and second vectors have a reporter gene. In yet other embodiments, the third vector further includes a positive selection marker which is integrated into the genome of the cells. In still other embodiments, the positive selection marker in the third vector (denoted the second positive selection marker) is operably linked to a prokaryotic promoter in the third cassette that integrates into the genome of the eukaryotic cells. In this case, the nucleic acid encoding a protein that modulates the activity of the regulatory element may be identified by (i) inserting a nucleic acid that includes the second positive selection marker and a segment of the eukaryotic genome flanking the third integrated cassette into bacterial cells under conditions that allow the selection of bacterial cells expressing the second positive selection marker under the control of the prokaryotic promoter, (ii) amplifying the segment of the eukaryotic genome that is inserted into the selected bacterial cells, and (iii) sequencing the amplified segment. In yet other embodiments, the positive selection marker in the third vector is operably linked to a yeast promoter in the third cassette that integrates into the genome of the eukaryotic cells. In this case, the nucleic acid encoding a protein that modulates the activity of the regulatory element may be identified by (i) inserting a nucleic acid that includes the second positive selection marker and a segment of the eukaryotic genome flanking the third integrated cassette into yeast cells under conditions that allow the selection of yeast cells expressing the second positive selection marker under the control of the yeast promoter, (ii) amplifying the segment of the eukaryotic genome that is inserted into the selected yeast cells, and (iii) sequencing the amplified segment.

In yet another aspect, the invention provides a method for treating, preventing, or stabilizing a disease that is mediated by, or associated with, a stimulatory agent. This method involves identifying a cell containing a regulatory element that is regulated by a stimulatory agent, selecting a compound that modulates the regulatory element or that modulates a protein which regulates the regulatory element, and administering the compound to a mammal having a disease or condition associated with the stimulatory agent or having an increased risk for the disease or condition. The cells that are regulated by stimulatory agent of interest may be identified using any of the methods of the invention.

In particular embodiments of the above aspect, the method involves inserting a vector which has a cassette including a positive selection marker, a negative selection marker, and a reporter gene into eukaryotic cells under conditions that result in the integration of the cassette into the genome of the cells such that the reporter gene is operably linked to a regulatory element in at least one cell. Cells in which expression of the reporter gene is specifically modulated by the stimulatory agent are selected. A compound that increases or decreases the effect of the stimulatory agent on the expression of the reporter gene is selected and administered to a mammal having a disease associated with the stimulatory agent. If the stimulatory agent is associated with an increased risk for the disease or is associated with increased severity of the disease, the administered compound preferably inhibits the ability of the stimulatory agent to modulate the expression of the reporter gene. Conversely, if the stimulatory agent is associated with a decreased risk for the disease or is associated with decreased severity of the disease, the administered compound preferably enhances the ability of the stimulatory agent to modulate the expression of the reporter gene.

In another embodiment of the above aspect, the method involves inserting a vector which has a cassette including a positive selection marker, a negative selection marker, and a nucleic acid segment encoding a transactivator polypeptide into eukaryotic cells under conditions that result in the integration of the cassette into the genome of the cells such that the nucleic acid segment encoding a transactivator polypeptide is operably linked to a regulatory element in at least one cell. Cells in which expression of the transactivator polypeptide is specifically modulated by the stimulatory agent are selected. A compound that increases or decreases the effect of the stimulatory agent on the expression of the transactivator polypeptide is selected and administered to a mammal having a disease associated with the stimulatory agent. If the stimulatory agent is associated with an increased risk for the disease or is associated with increased severity of the disease, the administered compound preferably inhibits the ability of the stimulatory agent to modulate the expression of the transactivator polypeptide. Conversely, if the stimulatory agent is associated with a decreased risk for the disease or is associated with decreased severity of the disease, the administered compound preferably enhances the ability of the stimulatory agent to modulate the expression of the transactivator polypeptide.

In still another aspect, the invention features a nucleic acid including a positive selection marker, a negative selection marker, and a reporter gene. In particular embodiments, the nucleic acid includes, in 5′ to 3′ order, a splice acceptor, a cassette including in any order a negative selection marker and a positive selection marker, a translation stop sequence, an internal ribosome entry site, a reporter gene, a translation stop sequence, and a polyadenylation signal. In another embodiment, the nucleic acid includes, in 5′ to 3′ order, a splice acceptor, a cassette including in any order a negative selection marker and a reporter gene, a translation stop sequence, a promoter, a positive selection marker, a translation stop sequence, and a polyadenylation signal. In other embodiments, the reporter gene is not operably linked to a promoter in the nucleic acid. In this embodiment, the nucleic acid may be inserted in a cell such that the reporter gene is operably linked to an endogenous promoter. In other embodiments, the nucleic acid also includes a nucleic acid segment encoding a transactivator polypeptide or also includes one or more recombinase signal sequences (e.g., LoxP sites).

In yet another aspect, the invention features a nucleic acid including a splice acceptor site and including a bacterial promoter operably linked to a positive selection marker or a reporter gene. In various embodiments, the nucleic acid also includes a negative selection marker which may or may not be operably linked to the bacterial promoter. In other embodiments, the nucleic acid also includes a translation stop sequence, an internal ribosome entry site, a reporter gene, a translation stop sequence, and a polyadenylation signal. In particular embodiments, the nucleic acid includes, in 5′ to 3′ order, a splice acceptor, a cassette including in any order a negative selection marker and a positive selection marker such that the positive selection marker is operably linked to a bacterial promoter, a translation stop sequence, an internal ribosome entry site, a reporter gene, a translation stop sequence, and a polyadenylation signal. In another embodiment, the nucleic acid includes, in 5′ to 3′ order, a splice acceptor, a cassette including in any order a negative selection marker and a reporter gene, a translation stop sequence, a bacterial promoter operably linked to a positive selection marker, a translation stop sequence, and a polyadenylation signal. In other embodiments, the positive selection marker is operably linked to the bacterial promoter, and the reporter gene is not operably linked to a promoter in the nucleic acid. In this embodiment, the nucleic acid may be inserted in a cell such that the reporter gene is operably linked to an endogenous promoter. In other embodiments, the nucleic acid also includes a nucleic acid segment encoding a transactivator polypeptide or also includes one or more recombinase signal sequences (e.g., LoxP sites). In still other embodiments, the nucleic acid includes a region of a eukaryotic genome, such as a region containing all or part of a gene or a regulatory element of interest or a region flanking a gene or a regulatory element of interest. Such nucleic acids enable bacterial cells to be used to facilitate the identification of trapped eukaryotic regulatory elements or genes of interest, as described herein.

In a related aspect, the invention features a nucleic acid including a positive selection marker, a negative selection marker, and a nucleic acid segment encoding a transactivator polypeptide. In various embodiment, the nucleic acid also includes one or more recombinase signal sequences (e.g., LoxP sites). In other embodiments, the nucleic acid segment encoding the transactivator polypeptide is not operably linked to a promoter in the nucleic acid.

In another related aspect, the invention provides a nucleic acid including a positive selection marker, a negative selection marker, and one or more recombinase signal sequences (e.g., LoxP sites).

In still another aspect, the invention features a nucleic acid including, in 5′ to 3′ sequence, an internal ribosome entry site, a nucleic acid segment encoding a transactivator polypeptide, a translation stop sequence; an internal ribosome entry site, a reporter gene; a translation stop sequence, and a polyadenylation signal. The nucleic acid may also include a recombinase signal sequence (e.g., a LoxP site). In another aspect, the invention provides a nucleic acid having a functional splice acceptor, a translation stop sequence, an internal ribosome entry site, a promoterless negative selection marker, a translational stop sequence, a polyadenylation signal, a promoter, positive selection marker, a translational stop sequence, and a polyadenylation signal. In other embodiments, the nucleic acid also includes an internal ribosome entry site, a nucleic acid segment encoding a transactivator polypeptide, a translation stop sequence, an internal ribosome entry site, a reporter gene, and a translation stop sequence. In yet other embodiments, the nucleic acid also includes an internal ribosome entry site, a nucleic acid segment encoding a transactivator polypeptide, and a translation stop sequence.

In another aspect, the invention features a vector, such as a retroviral vector that contains one or more nucleic acids of the invention. The vector may optionally include an integration sequence. In particular embodiments, the retroviral vector is a replication deficient viral vector, such as a SIN virus viral vector that contains a mutation in the 3′ LTR.

In yet another aspect, the invention features a cell (e.g., a eukaryotic or prokaryotic cell) containing a vector or nucleic acid of the invention. The cell may be responsive to only one or to more than one stimulatory agent. Exemplary cells contain (i) a first nucleic acid which includes a positive selection marker, a negative selection marker, and a nucleic acid segment encoding a transactivator polypeptide and (ii) a second nucleic acid which includes a promoter operably linked to a responsive element that is responsive to the transactivator polypeptide. In particular embodiments, the first nucleic acid also includes a reporter gene, or the second nucleic acid also includes a positive selection marker.

In a related aspect, the invention features a library of two or more cells (e.g., eukaryotic or prokaryotic cells) containing a vector or nucleic acid of the invention. In particular embodiments, the library of cells contains at least 5, 10, 20, 50, 100, 500, 1000, 50000, or more cells containing different trapped regulatory elements (e.g., different endogenous regulatory elements operably linked to a positive selection marker or reporter gene in a construct integrated into the genome of the cells) or different trapped genes (e.g., different endogenous genes operably linked downstream of a promoter in a construct integrated into the genome of the cells). Desirably, the library includes cells that are responsive to one or more stimulatory agents of interest. In other embodiments, the library includes 5, 10, 20, 50, 100, 500, 1000, 50000, or more different cells that are each responsive to a different stimulatory agent of interest.

In still another aspect, the invention features a screening method for selecting compounds that modulate the activity of a stimulatory agent of interest. This method includes contacting one or more cells of the invention that have a specific response to the stimulatory agent with one or more candidate compounds and the stimulatory agent. The candidate compounds which modulate (i.e., increase or decrease) the response to the stimulatory agent are selected.

In yet another aspect, the invention features a method for determining whether a candidate compound modulates the activity of a regulatory element of interest. The method includes contacting one or more cells of the invention that have the regulatory element of interest operably linked to a positive selection marker, reporter gene, or nucleic acid segment encoding a transactivator polypeptide with one or more candidate compounds. A candidate compound which modulates the expression of the positive selection marker, reporter gene, or nucleic acid segment encoding a transactivator polypeptide is selected, thereby selecting a candidate compound which modulates the activity of the regulatory element of interest. In particular embodiments, the modulation (i.e., increase or decrease) of the activity of the regulatory element of interest is associated with adverse side-effects of the candidate compound in vivo. In this case, the candidate compound is desirably eliminated from drug development due to the potential adverse side-effects (e.g., drug toxicity) of the candidate compound when administered to a mammal (e.g., a human). For example, a candidate compound that activates a regulatory element operably linked to a gene encoding an mRNA or a protein involved in a pathway associated with adverse side-effects is desirably eliminated from further drug development. In other embodiments, the method is used to determine whether particular combinations of two or more candidate compounds are likely to be associated with adverse side-effects when administered together (e.g., sequentially or concurrently) to a mammal. For example, a combination of candidate compounds that activates a regulatory element operably linked to a gene encoding an mRNA or a protein involved in a pathway associated with adverse side-effects is desirably eliminated from further drug development. In yet other embodiments, the method is performed prior to animal model studies or human clinical trials of the candidate compound or the combination of candidate compounds to determine whether or not the candidate compound(s) are likely associated with adverse side-effects prior to further drug development.

In various embodiments of any of the aspects of the invention, the cells are mast cells, stem cells, epithelial cells, fibroblast cells, cancer cells, lymphocytes, and liver cells. Other exemplary cells include cells from tumor cell lines from cancers of one of the following cell types: rectum, colon, ovary, prostate, pancreas, mammary gland, lung, ovary, kidney, cervix, tongue, thyroid, T lymphocyte, B lymphocyte, adenocarcinoma, small cell lung, burkitt's lymphoma, adenosquamous carcinoma, adrenocortical carcinoma, alveolar cell carcinoma, or hodgkin's lymphoma. An example of a eukaryotic genome is the genome of a mammalian cell. Suitable stimulatory agents include cytokines, growth factors, ligands, polypeptides, growth factors, antibodies, and chemical agents. Exemplary stimulatory agents include stem cell factor, IL-1, IL-3, IL-2, IL-6, IL-8, IL-18, IgE, Fibroblast Growth Factors (FGFs), FGF-1, FGF-2, FGF-3, transforming growth factor a (TGF-α), TGF-β, TNF-β, TNF-α, VEGF, leptin, epidermal growth factor (EGF), platelet-derived growth factor (PDGF), insulin, insulin-like growth factor-I& II, interferon-γ (IFN-γ), estrogen, testosterone, and colony stimulating factors (CSFs). It is also contemplated that the stimulatory agent controls the expression of an exogenous gene that is inserted into the cells. For example, the stimulatory agent (e.g., a chemical agent) can activate the promoter operably linked to an exogenous gene that encodes a protein which modulates the activity of an endogenous regulatory element of interest in the cells. In various embodiments, a protein activates the activity of an endogenous regulatory element of interest by inducing an activator or by inhibiting a repressor of the regulatory element. In other embodiments, the protein inhibits the activity of an endogenous regulatory element of interest by inhibiting an activator or by activating a repressor of the regulatory element. In other embodiments, the nucleic acid, cassette, vector, or cell includes a prokaryotic promoter (e.g., a bacterial promoter) or yeast promoter operably linked to a positive selection marker or reporter gene.

Desirable reporter genes encode an enzyme, such as secreted alkaline phosphatase, β-galactosidase, luciferase, and green fluorescent protein. In any of the above aspects, a nucleic acid segment encoding a single protein that has both positive selection traits and negative selection traits may be used as the positive and negative selection markers. In other embodiments, the negative selection marker and the positive selection marker encode different proteins. In still other embodiments, the reporter gene is different from the positive selection marker and/or the negative selection marker. Exemplary negative selection markers include nucleic acid segments encoding Hprt, gpt, HSV-tk, diphtheria toxin, ricin toxin, or cytosine deeaminase. Exemplary positive selection markers include nucleic acid segments encoding proteins conferring neomycin resistance, hygromycin resistance, histidinol resistance, xanthine utilization, Zeocin resistance, or bleomycin resistance. Examples of internal ribosome entry sites include mammalian, picornavirus, and polio internal ribosome entry sites.

### BRIEF DESCRIPTION OF THE DRAWINGS

FIG. 1 is a schematic diagram of a nucleic acid construct of this invention for use in preparing an induction gene trap vector. The following components are illustrated in the diagram: an internal ribosome entry site (IRES), a promoterless protein coding sequence coding a tetracycline regulator protein (“TetOn/Off”), an internal ribosome entry site, and a secreted alkaline phosphatase (“SEAP”).

FIG. 2 is a schematic diagram of a vector including the nucleic acid construct of FIG. 1 which also contains, upstream of the construct, the following additional components: a functional splice acceptor (SA), a translation stop sequence (“STOP”), an IRES site, and a promoterless protein coding sequence coding TK-ZEO; and a polyadenylation signal (“pA”) downstream of the nucleic acid sequence of FIG. 1.

FIG. 3 is a schematic diagram of another alternate vector for use in the invention which includes the nucleic acid construct of FIG. 1 and, upstream of the construct, a functional splice acceptor (SA), and a translation stop sequence (STOP), and downstream of the construct, an IRES site, a TK coding sequence, a phosphoglycerate kinase promoter (“PKG”), a ZEO coding sequence under the transcriptional control of the PKG promoter, and a polyadenylation signal (pA).

FIG. 4 is a schematic diagram of a nucleic acid construct of this invention for use in a vector in conjunction with the induction trap vectors of FIGS. 2 and 3. The following components are illustrated in the diagram: a minimal promoter sequence containing a tetracycline responsive element (“TRE_{Pcmv}”), a promoterless protein coding sequence encoding NEO, an IRES sequence, and a splice donor (“SD”).

FIG. 5 is a schematic diagram illustrating the use of a vector containing the construct of FIG. 1 and a vector containing the nucleic acid construct of FIG. 4 which can be integrated into the genomic loci to select genes that directly or indirectly regulate the genomic loci. A putative gene transcribed by the vector containing the nucleic acid construct of FIG. 4 is also shown.

FIG. 6 is a schematic representation illustrating the preparation of a ligand dependent cell from a selected cell line by contacting the cell with the transfection vector of FIG. 2, a physiological stimuli, and positive and negative selection drugs. The selection of specific cells which are activated or turned on by the physiological stimuli, and cells which are inactivated or turned off by the physiological stimuli, are illustrated in the left and right branches of the diagram, respectively.

FIG. 7 is a schematic representation illustrating an alternate method for the preparation of a ligand dependent cell from a selected cell line by contacting the cell with the transfection vector of FIG. 3, a physiological stimuli, and positive and negative selection drugs. The selection of specific cells which are activated or turned on by the physiological stimuli, and cells which are inactivated or turned off by the physiological stimuli, is illustrated in the left and right branches of the diagram, respectively. The production of SEAP is measured as an indicator of the response of the vector to the stimuli.

FIG. 8A is a schematic illustration of an induction trap vector. This vector includes a functional splice acceptor (SA), a translation stop sequence (STOP), an IRES site, a TK coding sequence, a ZEO coding sequence, a STOP sequence, a LoxP site, a IRES site, a SEAP coding sequence, a STOP sequence, a polyadenylation signal (PolyA), and a LoxP site. Other vectors that may be used in the methods of the invention include the corresponding induction trap vectors that contain only one LoxP site or that lack LoxP sites.

FIG. 8B is a schematic illustration of an exchange cassette that is used to replace the region of the induction trap vector of FIG. 8A that is flanked by LoxP sites, as described in Example 10. This cassette includes a LoxP site, an IRES site, a promoterless sequence encoding a tetracycline regulator protein (“teton/off”), a translation stop sequence (STOP), an IRES site, a β-galactosidase coding sequence (b-gal), a STOP sequence, a polyadenylation signal (PolyA), and a LoxP site. Other exchange cassettes that may be used in the methods of the invention include the corresponding cassettes with only one LoxP site. The LoxP site may be located in any part of the cassette.

FIG. 9A is a schematic illustration of a vector of the present invention. This vector contains a prokaryotic promoter operably linked to a positive selection marker (e.g., zeocin). This exemplary vector contains a functional splice acceptor (EN-2 SA), a translation stop sequence (STOP), an IRES site, a prokaryotic promoter, a negative selection marker (e.g., TK coding sequence), a positive selection marker (e.g., a ZEO coding sequence), another STOP sequence, a LoxP site, an IRES site, a reporter gene (e.g., a SEAP coding sequence), a STOP sequence, a polyadenylation signal (PolyA), and a LoxP site. The ClaI site represents a possible location of a unique restriction site in the vector. One skilled in the art would readily appreciate that the components of this vector may be present in different locations or different 5′ to 3′ arrangements. For example, the prokaryotic promoter may alternatively be located upstream of the first IRES site or between the TK coding sequence and the Zeo coding sequence. As noted above, the Zeo coding sequence may also be located upstream, instead of downstream, of the TK coding sequence. In some methods of the invention, the vector may lack the first LoxP site, the second IRES site, the SEAP coding sequence, the third STOP sequence, and/or the second LoxP site. Other vectors of the present invention contain a yeast promoter instead of the prokaryotic promoter in any of the vectors described above.

FIG. 9B is the polynucleotide sequence of an exemplary prokaryotic promoter, the T7 promoter (SEQ ID NO: 5). “RBS” denotes a ribosome binding site. Any other prokaryotic promoter or any yeast promoter may also be used in the nucleic acids, vectors, cells, and methods of the invention.

FIG. 10 is a schematic illustration of the uses of the cells of the invention to identify ligand specific pathways, redundant pathways, and pathways associated with toxic effects in vivo. This information is useful in the characterization of candidate drug products and the prediction of adverse side-effects caused by these products.

FIG. 11 is a schematic illustration of the use of the methods described herein to isolate EL4 or NIH3T3 fibroblast cells activated by TNFα or IL-1β.

FIG. 12A is a table confirming that the reporter gene (SEAP) that integrated into the genome of cells was integrated under the control of a regulatory element responsive to TNFα or IL-1β. FIG. 12B is a picture of a southern blot generated using a probe to the TK/Zeo selection markers in the integrated construct to confirm the integration of the construct in some of the selected NIH3T3 cell lines.

FIG. 13A is a schematic illustration of the identification of cells responsive to a single or multiple ligands. FIG. 13B is a bar graph illustrating the level of responsiveness of selected cell clones to IL-1β, TNFα, and IL-6.

FIGS. 14A and 14B are a set of bar graphs illustrating the level of responsiveness of selected clones to IL-1β, TNFα, PMA, and IL-10. FIG. 14C is a bar graph illustrating the level of responsiveness of selected clones to IL-1β, TNFα, SDF-1, MCP-1, and IL-10.

FIG. 15 is a graph illustrating the ability of the specific Cox-2 inhibitor, celecoxib, to inhibit the effect of IL-1β on SEAP reporter gene activity in selected NIH3T3 cells in a concentration dependent manner.

FIG. 16 is a graph illustrating the inability of celecoxib to significantly inhibit TNFα-induced SEAP reporter gene activity in selected EL-4 cells.

FIGS. 17A and 17B are a set of bar graphs illustrating the level of responsiveness to various ligands and ligand combinations in clone C-5 and clone PD6.

FIG. 18A is a graph illustrating the ability of the MEK inhibitor U0126 to inhibit the effect of IL-1β on SEAP reporter gene activity in selected NIH3T3 cells in a concentration dependent manner. As illustrated in FIG. 18B, cyclosporin A had a much smaller effect on SEAP activity in this assay.

### DETAILED DESCRIPTION OF THE INVENTION

The methods of this invention utilize cells, vectors, and stimulatory agents to generate cell lines, and to identify gene targets and regulatory elements which are useful for the selection of therapeutic agents from a library of drug candidates.

The cells which are useful in this invention are eukaryotic cells, preferably mammalian cells, and more preferably human cells. The eukaryotic cells are capable of differentiating into specific cell or tissue types, including both plant and animal cells and tissues. Particularly suitable are totipotent cells, such as stem cells, as well as mast cells, endothelial cells, epithelial cells, cancer cells, lymphocytes, and liver cells.

A reporter element useful in the nucleic acid constructs and vectors of this invention are elements which express indicators in cells which are capable of being detected using physical, chemical, or optical means. The detection can be visual, instrument assisted or completely automated. Suitable reporter elements include enzymes, such secreted alkaline phosphatase, luciferase, and green fluorescent protein. Enzymes which emit fluorescence can be detected using a luminometer.

An “induction gene trap vector” means a vector containing elements that allow for selection and insertion of the trap vector in an operably linked manner into an intron sequence of a regulated genomic loci of a cell, by techniques well known in the art, such as transfection, transduction, and the like, resulting in the transformation and integration of the vector into the genome. The induction gene trap vector contains a marker gene sequence expressing selection traits, such as positive and negative selection traits.

The induction gene trap vector may be “promoterless,” which means that the marker genes are not under the control of a promoter within the vector (although the vector may contain promoters which do not regulate these elements). In the case of a promoterless vector, regulation of the marker genes occurs as a result of endogenous regulatory elements or factors in the genome which respond to one or more exogenous stimulatory agents externally introduced into the cell.

Alternatively, the vector may contain a promoter for at least one component of the marker gene sequence, such as the PGK (phosphoglycerate kinase) promoter for the neo positive selection marker, as described in Mainguy et al., Nature Biotechnology, Vol. 18, pages 746-749 (2000). A vector containing a promoter for one such component, but lacking promoters for sequences expressing other selectable traits and for reporter sequences, is also included within the scope of this invention.

Elements or sequences in a vector which are “operably linked,” and vectors which are “operably integrated” into a genome, refer to nucleotide sequences which are linked, whether to encode an mRNA transcript of a desired gene product, or for regulatory control. “Operably linked” can also mean that selectable marker, transactivator, and reporter genes are encoded by the same transcription unit.

A “splice acceptor (“SA”), or functional splice acceptor, refers to a consensus sequence that permits the construct or vector to be processed such that it is included in a mature, biologically active mRNA, provided that it is integrated in an active chromosomal locus and transcribed as a contiguous part of the premessenger RNA of the chromosomal locus. Splice acceptors typically include the 3′ end of an intron and the 5′ end of an exon, while a splice donor (“SD”) typically includes the 5′ end of an exon and the 3′ end of an intron. Examples of these elements, as well as other gene elements used to prepare gene trap vectors, can be found in published patent application PCT/CA98/00667, Alberts et al., Molecular Biology of the Cell, page 373 (1994), and U.S. Pat. No. 5,922,601, the disclosures of which are incorporated herein by reference thereto in their entirety.

A translation stop sequence, or “STOP,” is a sequence that codes for translation stop codons in three different reading frames. The STOP sequence causes truncation of peptide chains encoded by exons upstream of the vector at the chromosomal locus and prevents the translational reading frame from proceeding into the selectable marker gene, thereby preventing translating in a non-sense reading frame.

An internal ribosome entry site, or “IRES,” as used herein, is an element which permits attachment of a downstream coding region or open reading frame with a cytoplasmic polysomal ribosome to initiate translation thereof in the absence of internal promoters. An IRES is included in the construct to initiate translation of selectable marker protein coding sequences. The encephalomyocarditis virus IRES is one such IRES which is suitable for use in this invention.

A “marker” refers to nucleotide sequences in vectors or genes encoding polypeptides or proteins which can be used to distinguish cells expressing the protein from those not expressing the protein. Marker genes can be detected using a variety of means and include selectable markers and assay markers. Selectable markers are genetic elements which can be selected or screened for when integrated into the genome or genomic loci of a cell. Selectable markers include markers having selection traits, such as drug resistant markers, antigenic markers, adherence markers, and the like. Examples of antigenic markers include those useful in fluorescence-activated cell sorting. Examples of adherence markers include receptors for adherence ligands that allow selective adherence Other selection markers include a variety of gene products that can be detected in experimental assay protocols, such as marker enzymes, amino acid sequence markers, cellular phenotypic markers, nucleic acid sequence markers, and the like.

The selectable markers also include markers with both negative and positive selection traits. In general, positive selection refers to the isolation of cells that express the marker gene, and negative selection refers to the isolation of cells that do not express the marker gene. In various embodiments, the expression of a negative selection marker leads to the selective elimination or death of cells containing the marker. A single gene or multiple genes can be used for positive and negative selection. Gene sequences which express a fusion protein having both positive and negative selection traits are preferred. As a specific example, a fusion protein can be expressed by a gene sequence encoding the negative selection marker Tk (thymidine kinase) and the positive selection marker neo (neomycin phosphotransferase). Details concerning gene markers having positive and/or negative selection traits and additional examples of selectable markers can be found in U.S. Pat. No. 5,922,601; filed Sep. 16, 1996; issued Jul. 13, 1999, the disclosure of which is incorporated by reference thereto in its entirety. Any of these selectable markers may also be used in the nucleic acid constructs and methods of the present invention.

A “transactivator” and a “transactivator polypeptide” are nucleic acid sequences and polypeptides, respectively, that transcribes, or causes the transcription of, a protein which effects the regulation of a genomic loci. Examples of transactivator polypeptides include transcription factors and growth factors. Other exemplary transactivator polypeptides include molecules involved in a signaling pathway. The transactivator polypeptides may directly or indirectly activate the transcription of a gene. For example, a transactivator polypeptide may directly bind a regulatory element; such as an enhancer, transcription factor binding site, or promoter; and activate the transcription of a gene downstream of the regulatory element. Alternatively, a transactivator polypeptide may activate another polypeptide that directly or indirectly activates transcription of the gene.

A “regulator unit or regulator protein” is a transactivator polypeptide that binds regulatory elements which effect the regulation of a genomic loci. For example, the transactivator polypeptide may bind a regulatory element (such as a tetracyline responsive promoter) and activate the transcription of an endogenous gene that is downstream of the regulatory element. The protein encoded by this endogenous gene may than activate a regulatory element of interest (such as an endogenous promoter or other regulatory element identified using an induction trap vector of the present invention).

A tetracycline regulator unit is an example of a transactivator regulatory sequence which expresses a protein (“tTA”) activated or repressed by tetracycline. The tetracycline regulator unit can be incorporated in a vector which acts in concert with a minimal promoter sequence containing tetracycline responsive elements, or “TRE_{Pcmv},” which is present in a complementary vector. See U.S. Pat. No. 5,464,758 and U.S. Pat. No. 5,814,618, the disclosures of which are incorporated herein by reference in their entirety. This pair of vectors can be operably integrated into the genome of a cell. When the cell is contacted with a stimulatory agent, the tetracycline regulator is turned on, causing the unit to generate a protein which binds to the minimal promoter sequence containing the tetracycline responsive element. This causes the minimal promoter to activate and induce transcription of genes downstream of the promoter. These transcribed genes can up regulate or down regulate the genomic loci, causing the tetracycline regulator unit to express more protein, thereby activating the promoter to transcribe additional copies of the gene, and so on. Eventually, as a result of this feedback process, enough genetic material is generated to be detected, isolated and sequenced.

“5′ RACE” cloning, as that expression is used herein, refers to 5′ rapid PCR amplification of cDNA ends (RACE). This procedure is described in detail by Skarnes et al., Genes and Development, 6, pages 903-918 (1992), the disclosure of which is incorporated herein by reference in its entirety.

By “cassette” is meant a segment of a nucleic acid.

By “polypeptide” is meant a sequence of two or more covalently bonded naturally-occurring or modified amino acids. The terms “peptide,” “polypeptide,” and “protein” are used interchangeably herein.

The method and vectors of this invention can be used to select cell lines and identify regulatory components which respond to stimulation from a selected stimulatory agent. This is accomplished by utilizing induction gene trap vectors to introduce specific polynucleotide sequences into genomic loci which respond to selected stimuli. While certain specific nucleotides and vectors have been illustrated herein, this is done for convenience in understanding the invention only and is not intended to limit the scope of the invention. Other vectors can be readily designed by those skilled in the art and advantageously used to practice the methods described herein.

In general, the induction trap vectors of this invention contain a transactivator gene or polypeptide coding sequence, and/or contain a reporter element or sequence. The reporter element is preferably a sequence encoding an enzyme which is capable of being detected. Suitable enzymes are well known in the art and include secreted alkaline phosphatase (SEAP), Luciferase™ and green fluorescent protein. Enzymes emitting light can be detected using, for instance, a fluorescent activated cell sorter or similar device.

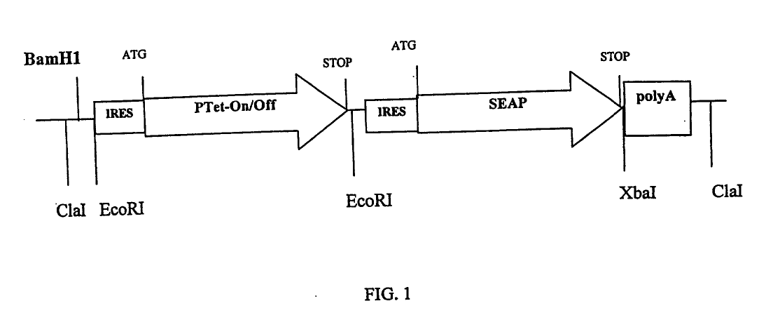

One specific nucleic acid construct is shown in FIG. 1. This construct can be incorporated in an induction gene trap vector and used to transfect cells of interest. As shown in FIG. 1, some constructs which are operable in this invention include a cassette containing an internal ribosome entry site, a transactivator gene such as a promoterless protein coding sequence coding a tetracycline regulator protein, an internal ribosome entry site, and a reporter sequence such as secreted alkaline phosphatase. Obviously, other transactivator genes and reporter elements can be used in the construct in place of the specific components shown in the FIG. 1. Moreover, a translation stop sequence can be inserted between the tetracycline regulator unit and the IRES sequence, and a STOP sequence can be used at the end of the construct as well. The construct can be incorporated into a vector, such as a viral vector, for use in transfecting cells.

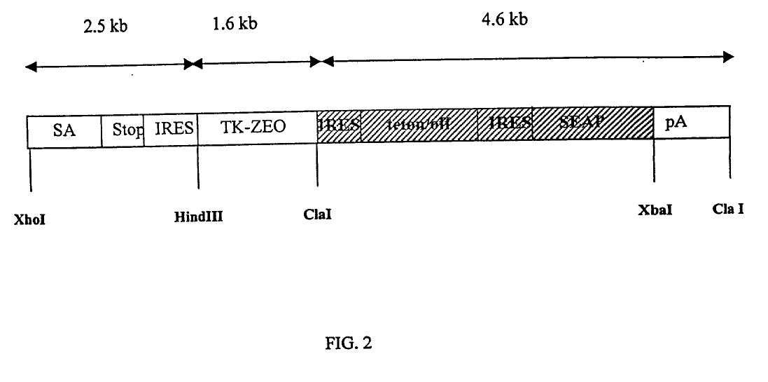

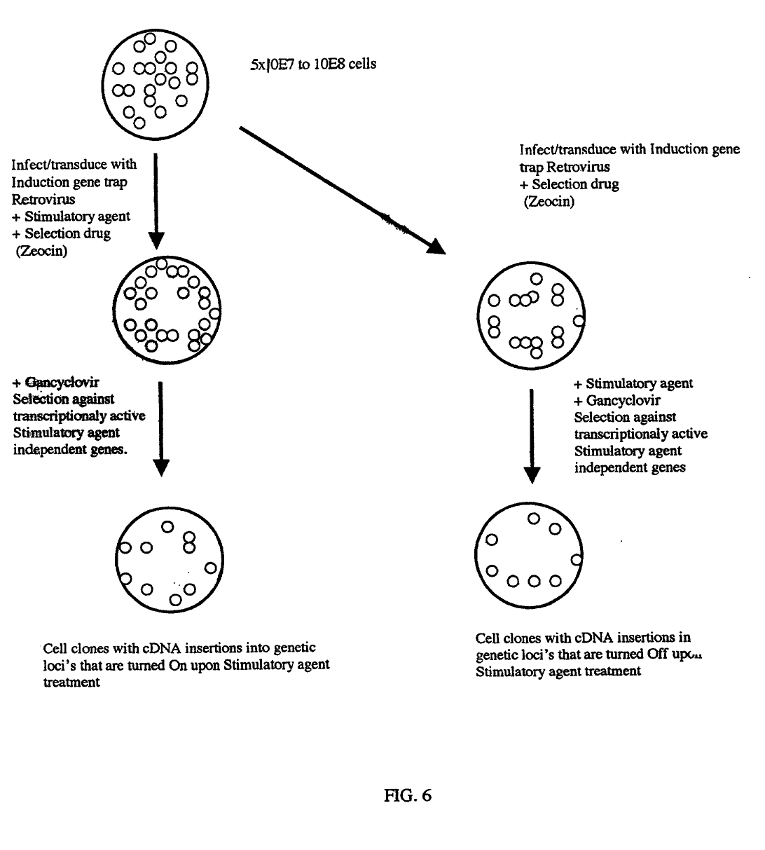

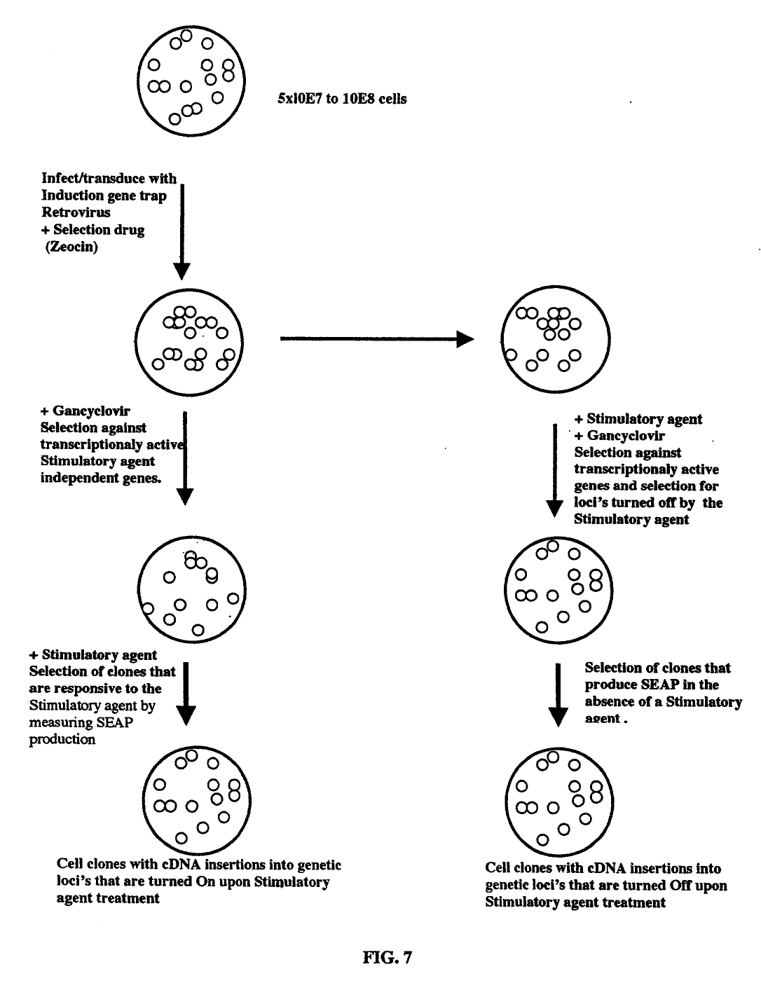

Another vector which is useful in this invention is illustrated in FIG. 2. The vector of FIG. 2 contains the following operably linked components: a functional splice acceptor; a translation stop sequence; an IRES site; and a promoterless protein coding sequence encoding a fusion protein having positive and negative selection traits, such as the gene encoding the fusion protein for the negative/positive selection polypeptide Tk-Zeo; an internal ribosome entry site; a gene marker such as a promoterless protein coding sequence encoding a tetracycline regulator protein, an internal ribosome entry site; a reporter sequence such as a sequence encoding secreted alkaline phosphatase; and a polyadenylation signal. Some components of this vector may be redundant depending on the particular uses of the vector. For instance, if the vector is used to select for a cell line responsive to a stimulatory agent, it may be possible to eliminate the reporter element, and its associated IRES, depending on the particular selection protocol used in the gene trap procedure, as illustrated in FIGS. 6 and 7.

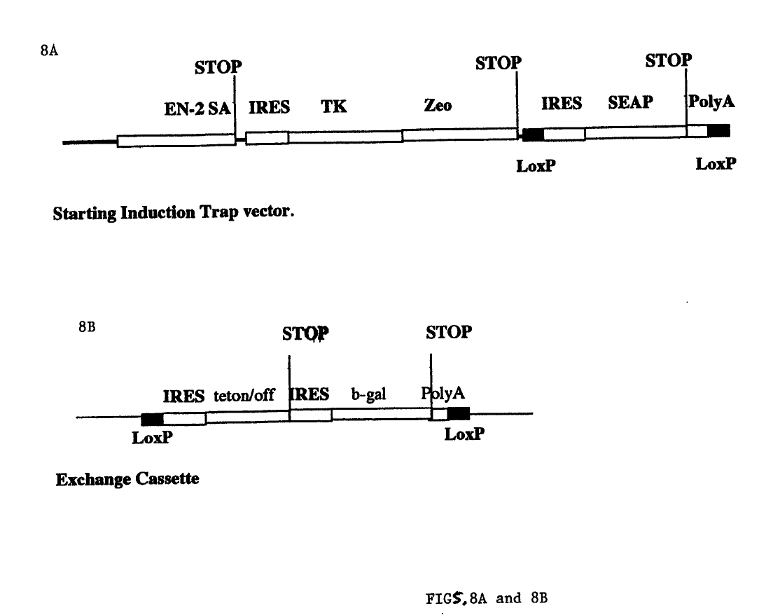

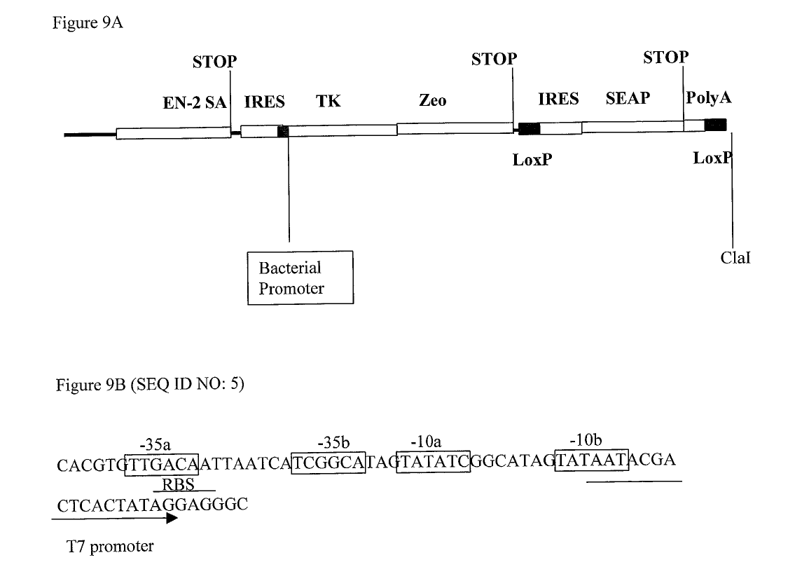

The vector illustrated in FIG. 2, FIG. 8A, or FIG. 9A can be used to generate cell lines using the procedure outlined in FIG. 6. As shown, cells are transformed, using suitable techniques such as transfection or transduction, with the retroviral vectors of FIG. 2. A stimulatory agent, such as IL-3, and a selection drug, such as zeocin, are added to the cell culture medium. The live cells remaining in the culture medium are cells with the vector integrated into the genomic loci that are (1) turned on (activated) by the stimulatory agent and (2) contain activated housekeeping genes. The live cells are separated from the medium and placed in a fresh medium with gancyclovir to eliminate the cells with active housekeeping genes. Cells that are turned on by the stimulating agent are identified and isolated.

Alternatively, the cells in FIG. 6 are transformed with the retroviral vectors of FIG. 2, FIG. 8A, or FIG. 9A. A selection drug (zeocin) is added to the medium, and those cells remaining are cells containing housekeeping genes, and cells which are turned on in the absence of the stimulatory agent. A stimulatory agent, such as IL-3, and gancyclovir are added to fresh medium containing the activated cells, and cells which contain activated housekeeping genes are eliminated. The cells remaining are those cells which are turned off by the stimulatory agent.

FIG. 2 illustrates another vector which can be used to generated cell lines. This vector includes the following operably linked components in downstream sequence: a functional splice acceptor sequence, a translation stop sequence, an internal ribosome entry site, a transactivator gene such as a promoterless protein coding sequence coding a tetracycline regulator protein, an internal ribosome entry site, a reporter sequence such as secreted alkaline phosphatase, an IRES site, a coding sequence encoding TK, a phosphoglycerate kinase promoter (“PKG”), a Zeocin coding sequence under the transcriptional control of the PKG promoter, and a polyadenylation signal (pA).

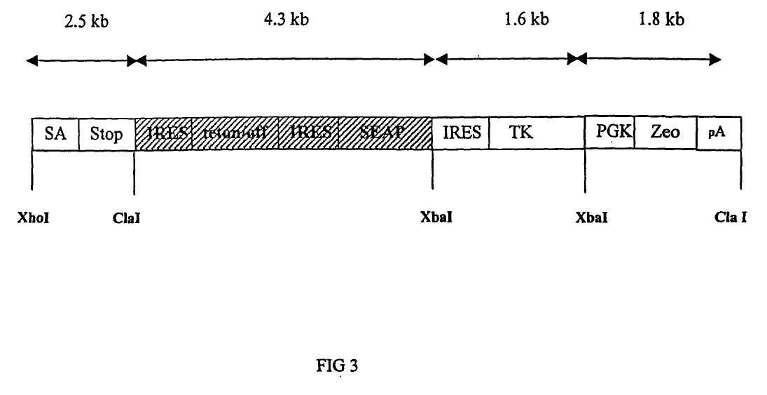

The vector illustrated in FIG. 3, FIG. 8A, or FIG. 9A can be used to generate cell lines using the procedure outlined in FIG. 7. As shown in FIG. 7, cells are transformed with the induction gene trap vector shown in FIG. 3, FIG. 8A, or FIG. 9A and a selection drug (e.g., zeocin) is added to the medium to eliminate cells which do not have the vector integrated into the genomic loci. The medium is changed, and gancyclovir is added to eliminate cells containing active housekeeping genes. A stimulatory agent is added, and those cells which respond to the agent are selected based on the amount of secreted alkaline phosphatase produced by the cells. These are cells which are activated by the stimulatory agent.

Alternatively, the cells in FIG. 7 are transformed with the retroviral vector of FIG. 3, and a selection drug (zeocin) is added to the medium. To eliminate cells which do not have the vector integrated into the genome. The medium is changed, and gancyclovir and a stimulatory agent are added to eliminate cells which contain housekeeping genes. Cells which are turned off by the stimulatory agent are selected by measuring the amount of secreted alkaline phosphatase produced by the cells in the absence of the stimulatory agent.

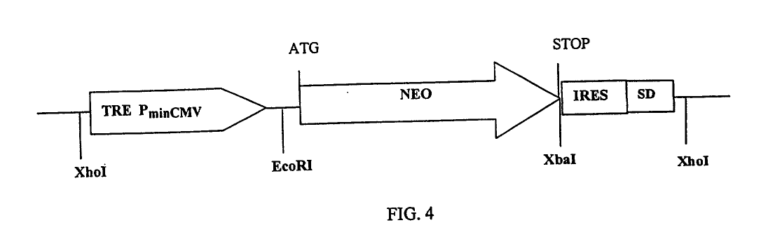

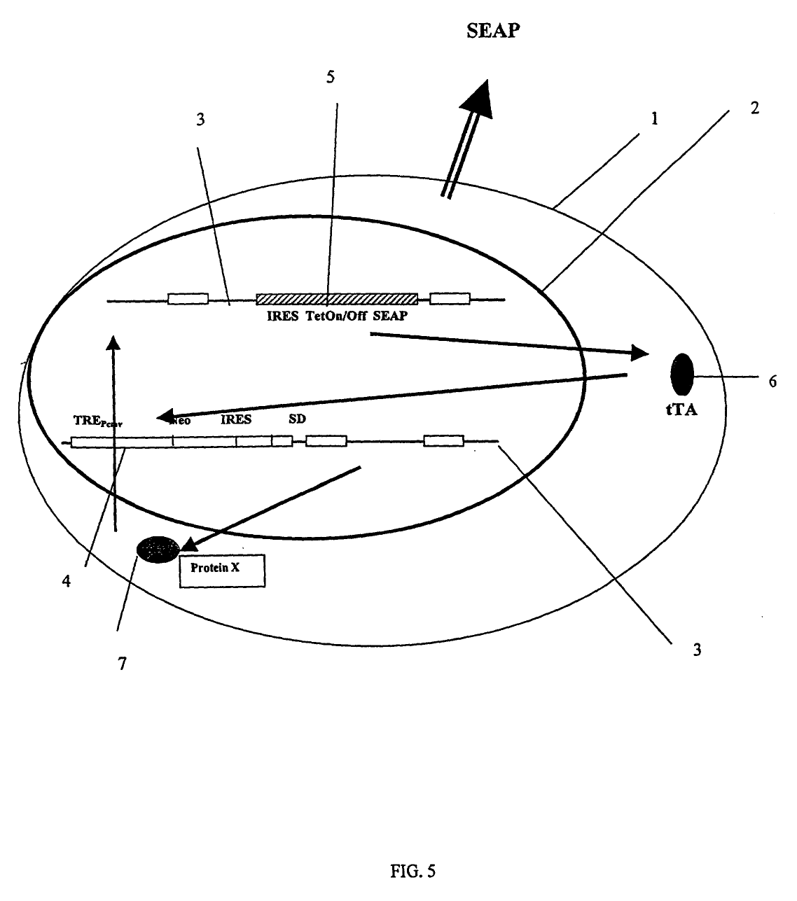

FIG. 4 illustrates a vector which can be used in combination with a vector containing the nucleic acid sequence shown in the shaded area depicted in FIG. 2 or FIG. 3 to identify a gene which is capable of up regulating or down regulating the genomic loci of a cell which responds to a stimulatory agent, such as the cells identified in FIGS. 6 or 7. The vector of FIG. 4 includes the following components operably linked in downstream sequence: a minimal promoter sequence containing a tetracycline responsive element; a promoterless protein coding sequence encoding Neo; an IRES sequence; and a splice donor. The shaded area in FIGS. 2 or 3 contains the following components in downstream sequence: an IRES sequence, a Tet On/Off sequence, an IRES sequence, and an SEAP expression sequence. Alternatively, a region of the induction trap vector of FIG. 8A may be replaced with the exchange cassette of FIG. 8B to generate a vector 5 that includes an IRES sequence, a TetOn/Off sequence, a STOP sequence, an IRES sequence, a galactosidase expression sequence, a STOP sequence, and a polyadenylation sequence. The secondary cell infection procedure is illustrated in FIG. 5.

As shown in FIG. 5, cell 1 having a nucleus 2 is transfected with vectors 4 and 5, and the vectors are integrated into the genomic loci 3. Vector 4, which can be the vector of FIG. 4, and vector 5, which can be a vector containing the nucleic acid sequence shown as the shaded area in FIGS. 2 or 3. are transfected and integrated into the genomic loci 3 of cell 1. Cell 1 can be a cell of the type depicted in FIG. 6 or FIG. 7. The integrated vectors act in a complementary fashion to cause an increase in the expression of a gene downstream of the integration site of the transfected vector 4, and the expression of the tetracycline regulator protein coded by vector 5. This occurs as a result of the activation of the tetracycline regulator unit that transcribes protein 6 (tTA), which is a protein activated or repressed by tetracycline. The tTA protein 6 binds to the minimal promoter sequence containing the tetracycline responsive element in vector 4, activating the TRE_{Pcmv} promoter and transcribing additional protein 7 (protein X) of the downstream gene. The protein 7 transcribed by the downstream gene up regulates or down regulates the genomic loci 3, causing increased expression of the tetracycline regulator unit in vector 5, thereby activating the TRE_{Pcmv} promoter in vector 4 to transcribe additional protein 7 from the downstream gene. This process repeats itself in a continuous loop until sufficient protein 7 is transcribed to permit the collection and identification of protein 7 or its mRNA. This allows the corresponding gene to be identified and characterized. As shown in FIG. 5, the gene transcription process can be monitored by the production of SEAP by vector 5 (or by the production of β-galactosidase by the exchange cassette of FIG. 8B) in response to the indirect or direct regulation of the primary genetic loci 3.

The isolation and identification of trapped regulatory elements as described herein allows the identification of genes operably linked to the trapped regulatory elements and thus the identification of genes whose transcription is increased or decreased by a stimulatory agent of interest. The regulation of these genes can be compared under different environmental conditions and in different cell lines (e.g., cells from different tissues, different organisms, or different disease animal models) to determine whether the genes are regulated the same way in various cell types and to determine whether the regulation of the genes is altered in the presence of certain environmental factors or disease states. The selected cells may be further characterized to determine what proteins affect the transcription of the genes (as described in Example 10) or to determine the role of the encoded proteins in vivo, such as the role of wild-type or mutant forms of the encoded protein in inhibiting, causing, or enhancing a disease state.

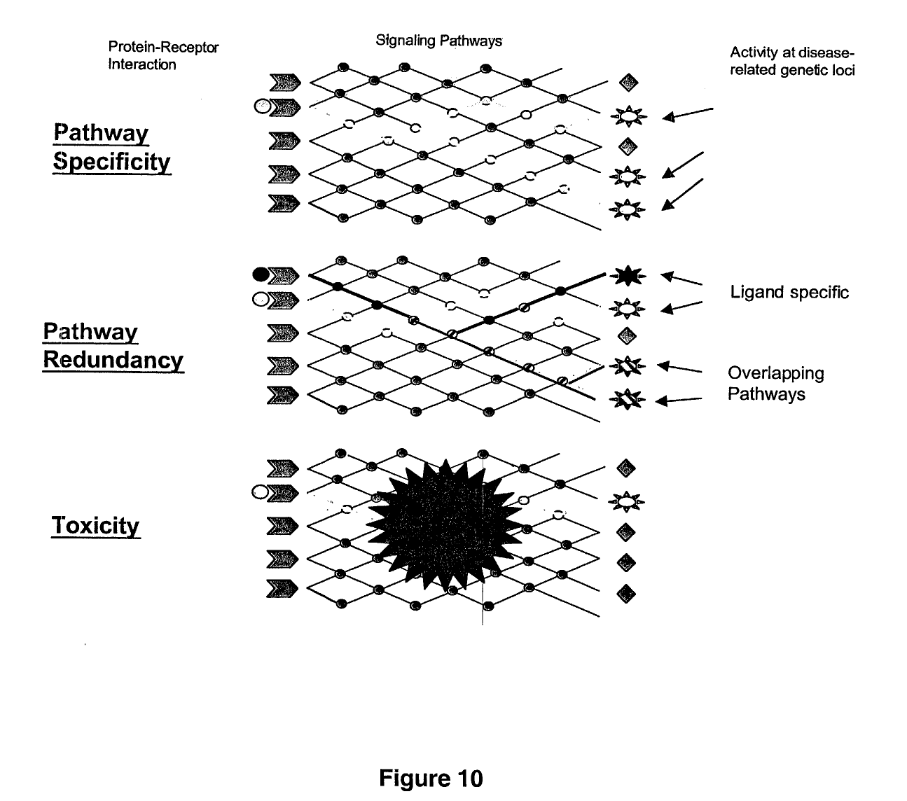

The selected cells may also be classified based on the characteristics of the trapped regulatory elements or trapped genes. For example, cells containing trapped nucleic acids associated with the expression of proteins in a common class of proteins (e.g., kinases, phosphatases, proteins in the same signal transduction pathway, or proteins associated with the same disease state) may be classified into the same group. A group of cells may be contacted with a candidate compound such as a potential drug product to compare the effect of the candidate compound on each cell, thereby determining whether the affect of the candidate compound is specific for certain trapped nucleic acids or has a general effect on multiple trapped nucleic acids. As illustrated in FIG. 10, the cells can be used to determine whether different ligands act through separate or overlapping pathways.

The cells of the invention are also useful in the identification and validation of new genetic targets for the treatment or prevention of diseases. For example, the cells can be used to determine whether activation or inhibition of a trapped regulatory element or gene of interest modulates a pathway associated with a disease state. These cells can be used in screening assays to identify new drug products or lead compounds for drug development. Cells containing an inserted reporter gene can be used to identify regulatory elements or promoters that are responsive to a pharmaceutically active compound, such as TNF-α. Cell lines may be selected that are responsive to only TNF-α or are also responsive to other pro-inflammatory cytokines. For example, cell clones which respond to both pro-inflammatory cytokines, TNF-α and IL-1β, can be selected by treating TNF-α cell lines with IL-1β in the presence of the positive selection drug. Thus, by establishing a broad library of cell lines, each incorporating a reporter gene at regulated genetic sites and exhibiting a standardized read-out mechanism, a platform can be assembled with the capability to readily test for efficacy and side-effects of compounds targeting regulatory pathways.

Therapeutic agents based on the identified gene (e.g., antisense molecules, gene activators, or gene inhibitors) can then be appropriately devised. For instance, the gene can be used in gene therapy applications when formulated into appropriate vectors tolerated by the patient in a medical therapeutic delivery vehicle. Alternatively, the gene or its regulatory elements, such as promoters and enhancers, can be used as drug targets to identify potential therapeutic candidates from libraries of compounds.

The following examples are illustrative of certain embodiments of the invention, and are intended to further describe the present invention, without limiting it thereby. Various modifications can be made to these embodiments without departing from the spirit or scope of the invention.

### EXAMPLE 1

### Preparation of a Nucleic Acid Construct

The retroviral vector containing the insert shown in FIG. 1 is prepared in 5 steps. These steps involve the transfer of cDNA fragments coding for the SEAP and the tTA into expression vectors containing IRES, and then the subsequent merger and transfer of these two constructs into a retroviral vector. These steps are as follows:

Step 1: The SmaI-XbaI fragment from the pSEAP-2 vector (Clontech) is isolated and inserted by ligation into the SmaI-XbaI sites of the vector pIRES (Clontech).

Step 2: The EcoRI-BamHI fragment from the pTet-on plasmid (Clontech) is inserted by blunt end ligation into the SmaI site of the vector pIRES.

Step 3: The EcoRI-XbaI fragment from the vector constructed in step 2 is transferred by bluntend ligation into the SmaI site of the vector PBSKS (Stratagene).

Step 4: The EcoRI-EcoRI fragment of the vector constructed in step 3 is transferred into the EcoRI site of the vector constructed in step 1.

Step 5: The ClaI-ClaI fragment resultant from step 4 is transferred into the ClaI site of the retroviral vector pSIR (Clontech).

### EXAMPLE 2

### Construction of a Vector Containing the Nucleic Acid Construct of Example 1

This vector is constructed in two steps that include replacement of the neomycin resistance gene in the “SATEO” construct (U.S. Pat. No. 5,922,601) by the Zeocin resistance gene, and its subsequent transfer to the retroviral vector described in Example 1. These steps are:

Step 1: Isolation of Zeo cDNA EagI-EcoRI fragment from the pEM7/Zeo vector (Invitrogen) and ligation into the Eag I-EcoRI sites of SATEO.

Step 2: Isolation of XhoI-BamHl fragment from the construct made in step 1, and its ligation into the XhoI-BamHl sites of the vector described in Example 1.

Retrovirus is produced by transfection into the helper 293 packaging cell line as described in the Clontech Manuel for the pSIR vector. Retroviral titer is established by measuring the amount of SEAP activity in infected 3t3 fibroblast cells.

### EXAMPLE 3

### Construction of an Alternative Vector

This vector is constructed in three steps involving the initial deletion of the neomycin resistance gene from the “SATEO” construct (U.S. Pat. No. 5,922,601), and the transfer of the resultant insert in combination with the insert made in step 4 of Example 1 into the pSIR vector. The steps are:

Step 1: Removal of EagI-SalI insert by digestion “SATEO” construct (U.S. Pat. No. 5,922,601), and followed by bluntend ligation.

Step 2: Transferring the ClaI-ClaI fragment from the construct made in step 4 of Example 1 to the EcoRI site of the vector pSIR vector.

Step 3: Isolation of XhoI-BamHl fragment from construct made in step 1, and its ligation into the XhoI-BamHl sites of the vector described step 2.

Retrovirus is produced and tittered as described in Example 2.

### EXAMPLE 4

### Preparation of Nucleic Acid Construct for Identifying Genes

This construct is synthesized in 5 steps. The first step involves the synthesis and transfer of an IRES-SD fragment and its placement down stream in a neomycin resistance gene. The entire insert is then transferred into the pTRE vector. The pTRE vector and the insert are then transferred into a retroviral vector. The steps are involved are as follows:

Step 1: Two complementary oligonucleotides containing the SD site flanked by restriction sites of XbaI-NotI are synthesized: 5′-aatctagaaggtaaggcggccgcaa-3′ (SEQ ID NO.: 1) and 5′-ttgcggccgccttaccttctagatt-3′ (SEQ ID NO.: 2)

Step 2: Oligonuclotides described in step 1 are annealed and cut by restriction enzymes before being ligated into the XbaI-NotI site in the pIRES vector (Clontech).

Step 3: The neomycin gene from the psv2neo construct (Stratagene) is ligated by bluntend into the MluI site of the vector constructed in step 2.

Step 4: The EcoRI-BamHI fragment of the vector from step 3 is isolated and ligated into the EcoRI-BamHI site of the pTRE vector (Clontech).

Step 5: The XhoI-NotI fragment from the construct synthesized in step 4 is transferred into the XhoI-BamHI site of the pSIR retroviral vector (Clontech).

### EXAMPLE 5

### Preparation of Mast Cell Line library

Mast cells are known to play a central role in inflammatory diseases such as asthma. Cytokines, such as Stem cell factor (SCF) and IL-3, are known to be critical for the proliferative and activation response of mast cells. In vivo, these cytokines induce not only the accumulation of mast cells in airways, but also prime the cells and enhance their hyper-responsiveness. The identification of regulatory factors that can modulate mast cell responses by such cytokines are prime targets for inhibitory drugs. Furthermore, identification of regulatory factors that are involved in the regulation of more than one cytokine in mast cells is likely to represent a critical convergent point of different important pathways. Generation and identification of a mast cell line incorporating such regulatory factors would therefore be highly useful for both high throughput screen for inhibitors and as a means for gene discovery.

Experimental Procedure

The human mast cell line HMC-1 is an established cell line that manifests proliferative and activation responses to various cytokines including IL-3 and SCF. Treatment of cells with cytokines can either up or down regulate genes. In this experiment, cell lines are established containing genes that are up-regulated by IL-3 and SCF. (HMC-I cells are normally maintained in culture medium without additional growth factors).

To trap IL-3 responsive regulatory regions of genes, HMC-1 cells are cultured in medium without growth factor supplement overnight for 12 hours. HMC-I cells are then cultured in IL-3 containing medium for 6 hours. After this, the cells are infected with a retrovirus carrying the induction gene trap vector described in Example 2 by culturing cells in viral-containing medium for 12 hours. Infected cells are washed once and redistributed into 96 well culture plates at cell numbers of 5000-10,000 per 100 ul per well. Selection is initiated with zeocin-containing medium. After three days, surviving cells are collected. These cells represent 1) house-keeping genes or 2) genes activated by IL-3 resulting in the promoters driving production of reporter ZEO, and reporter gene transcripts and protein. Reporter assays are performed to demonstrate and confirm the specific expression in the surviving clones.

Selection of the IL-3 responsive genes demonstrates the reversibility of IL-3 induction by switching the culture medium to IL-3-minus medium supplemented with gancyclovir. Housekeeping genes that continue to be active are selected against by the expression of thymidine kinase resulting in the elimination of these clones. Surviving clones represent IL-3 responsive genes. To confirm this, a reporter assay are repeated 12 hours after IL-3 deprivation. Clones that are reporter negative are identified.

A similar experiment as described above is carried out to establish cell lines that are SCF responsive. A similar experiment is carried out except that IL-3 is used in place of SCF.

To confirm the factor-responsiveness of the isolated clones, reporter assays are repeated for each clone before and after induction. The results are further strengthened with titration curves to quantify dose response.

Each IL-3-responsive cell line is tested with SCF to identify cell lines that will respond to both cytokines. Similarly, SCF-responsive cell lines are tested with IL-3.

In the final step, the identity of the gene for each clone is established. Primers have been synthesized that are specific for use in a 5′ RACE with the vectors of this invention to allow cloning and sequencing of the trapped gene. From this information, clones that are responsive to 2 or more factors will be identified.

### EXAMPLE 6

### Preparation of Endothelial Cell Line library

Inhibition of angiogenesis is a potent approach to eliminate cancerous tissues. Currently, an increasing number of “anti-angiogenic” molecules have been isolated and are in clinical trials. However, their effect on human cancers has not been established. It is also not known whether a single angiogenesis inhibitor will suffice to maintain the persistent suppression of cancer growth. It is likely that eventually, a combination of therapeutic inhibitors may be necessary. This is not surprising since extensive experimental data has demonstrated that several factors have the capacity to induce proliferation of endothelial cells and promote the genesis of new blood vessels. These factors originated both from the cancer cells and the surrounding stromal components. Gene products modulating these events represent prime targets for inhibitory drugs and small molecules. Similarly, the group of genes that are responsive to more than one factor likely represent critical convergent points of different important pathways. Such cell lines would therefore represent a highly useful tool in a high throughput screen for inhibitors.

Experimental procedure

Several well studied factors are known to induce endothelia cell growth. Clones responsive to VEGF, TGF-β and FGF-2 are established. The human endothelial line ECV304 or HMEC1 that has been extensively used in other experiments is utilized. Endothelial cells are plated out in 96 well plates at sub-confluent cell density. Cells are stimulated with VEGF-containing medium for 6 hours followed by infection with medium containing retroviral-vectors as described above 12 hours after initiation of infection, the culture medium is replaced with zeocin-containing medium to select for trapped active genes. As described above, after three to four days of selection, surviving cells represent 1) house-keeping genes or 2) genes induced by VEGF. Reporter assays are performed to demonstrate and confirm the specific expression of the reporter gene in the surviving clones.

To select for VEGF-responsive regulatory regions of genes, the reversibility of VEGF induction is demonstrated by switching the culture medium to medium without VEGF, and supplemented with gancyclovir. Reporter assays are performed 12 hours after VEGF deprivation. Clones that become reporter-negative are identified. Reporter-positive clones representing housekeeping genes that continued to be actively transcribed are selected against by the expression of thymidine kinase resulting in the elimination of these clones. Surviving clones after 3-4 days represent VEGF-responsive genes.

To establish cell lines that are TGF-β or FGF-2 responsive, a similar experimental sequence as described above for VEGF is carried out, except that TGF-β or FGF-2 is used in place of VEGF.

To confirm the specific factor-responsive characteristic of the isolated clones, reporter assays are repeated for each clone before and after induction. Using primers as discussed in previous section in 5′ RACE analysis, the identity of the trapped genes is established. Clones that represent genes in endothelial cells responsive to all three stimulants are thus identified.

### EXAMPLE 7

### Selection of a Gene Which up Regulates or Down Regulates the Selected Loci

Selected clones from Examples 5 and 6 are cultured in a growth medium until 80% confluency is reached. These cells are then infected with a retrovirus carrying the gene trap vector described in Example 4 by culturing cells in viral containing medium for 12 hours. Infected cells are washed once and redistributed into 96 well culture plates at cell numbers of 5000-10,000 per 100 ul per well. Selection is initiated with G418-containing medium. After three days, surviving cells are collected. These cells represent cells in which the viral vector has been successfully integrated. The clones from Example 5 are placed in growth containing medium containing Zeocin and G418. This allows for the selection of cells with active genomic loci, in addition to an integrated gene trap vector which confers resistance to G418. Reporter assays are performed to demonstrate and confirm the specific expression in the surviving clones.

The clones from Example 6 are selected based on testing for ligand independent SEAP activity. For example, these cells may be incubated in the absence of the stimulatory agent to identify trapped genes that encode proteins which modulate the activity of the regulatory element operably linked to the SEAP coding sequence in a ligand independent manner.

The cells may also be incubated in the presence of the stimulatory agent to identify trapped genes that encode proteins which modulate the activity of the regulatory element in a ligand dependent manner. In particular, the encoded proteins that are more active in the presence of the stimulatory agent produce a greater effect on the level of SEAP activity in the presence of the stimulatory agent. These encoded proteins may be directly activated by the stimulatory agent or may be activated by another protein which is directly or indirectly activated by the stimulatory agent. Alternatively, the stimulatory agent may inhibit another protein that would otherwise inhibit the protein encoded by the trapped gene. The encoded proteins that are less active in the presence of the stimulatory agent produce a smaller effect on the level of SEAP activity in the presence of the stimulatory agent. These encoded proteins may be directly or indirectly inhibited by the stimulatory agent.

Validity of the model is tested by looking for clones that demonstrate SEAP production in a tetracycline-dependent manner. For example, trapped genes that encode proteins which activate a regulatory element of interest enhance the production of SEAP in the presence of tetracycline. Conversely, trapped genes that encode proteins which inactivate a regulatory element of interest inhibit the production of SEAP in the presence of tetracycline. Using primers corresponding to sequences upstream of the SD site in the gene trap vector in 3′ RACE analysis, the identity of the trapped genes is established.

### EXAMPLE 8

### Screening for Inhibitors and Antagonists

Selected cell clones from Examples 6 and 7 are used directly to screen a natural products library (EXALPHA) for inhibitors and activators of SCF and VEGF activity. For identification of inhibitors to the SCF mediated signaling in the HMC-1 clones, these cells were cultured and plated equally into eleven 96 well plates. For identification of inhibitors and activators, a 1 nM aliquot from each well of the natural products library were transferred to each well of cultured cells in the presence of SCF. The amount of SEAP activity is measured and compared in well-to-well manner. For identification of SCF independent activators, this screen is done in the absence of SCF.

For identification of inhibitors and activators of the VEGF mediated signaling in ECV304 cells, similar techniques as above are used. ECV304 cell clones are harvested and plated in 96 well plates, and the relative amount of SEAP produced is compared in each well in the presence of 1 nM of the natural product fraction and VEGF.

### EXAMPLE 9

### Identification of Regulatory Elements that are Responsive to a Stimulatory Agent

For the identification of regulatory elements that are responsive to a stimulatory agent of interest, cells are infected with a retrovirus carrying the induction gene trap vector illustrated in FIG. 8A or FIG. 9A or a similar vector containing one or no LoxP sites, as described in Example 2. The infected cells are then washed once and redistributed into 96 well culture plates.

Selection of Regulatory Elements that are Activated by a Stimulatory Agent

To identify regulatory elements (e.g., enhancers or promoters) that are activated by a stimulatory agent of interest, the cells are incubated in the presence of the stimulatory agent of interest and Zeocin, the positive selection drug (FIG. 6). This step results in the isolation of cells in which the construct has stably integrated into the genome under the control of a promoter that may or may not be regulated by the stimulating agent. To eliminate cells in which the construct is expressed under the control of a promoter that is not regulated by the stimulating agent (e.g., a housekeeping gene promoter), the cells are cultured in the absence of the stimulating agent, but in the presence of gancyclovir. This step eliminates cells that express the negative selective marker thymidine kinase in the absence of the stimulatory agent and results in the isolation of desired cells in which the construct has stably integrated into the genome under the control of a promoter (or other regulatory element) that is regulated by the stimulating agent. While not meant to limit the invention in any way, it is noted that the integration of the construct into the cells is an essentially random event, thus not all of the cells will contain a construct integrated under the control of a endogenous promoter or under the control of an endogenous promoter modulated by the stimulating agent of interest.

Another method that may be used to identify regulatory elements that are activated by the stimulatory agent involves first incubating the cells with Zeocin to select cells containing the induction trap vector (FIG. 7). The selected cells are then incubated in the presence of gancyclovir without the stimulatory agent. This step eliminates undesired cells in which the trapped regulatory elements are transcriptionally active in the absence of the stimulatory agent. The remaining cells are incubated in the presence of the stimulatory agent. The desired cells that are responsive to the stimulatory agent are selected based on the transcription of the reporter gene (e.g. by measuring SEAP production). Thus, the reporter gene from the induction trap vector allows the effect of the stimulatory agent on the regulatory element to be quantitated. This quantitation allows the effect of different stimulatory agents on the same cell to be compared and allows the effect of one stimulatory agent on different cells to be compared. In desirable embodiments, the effect of a stimulatory agent of interest is at least 2, 5, 8, 10, 20, 50, or 100 fold greater than the effect of another stimulatory agent on the transcription of the reporter gene. In other embodiments, the effect of a stimulatory agent of interest is at least 2, 5, 8, 10, 20, 50, or 100 fold greater than the effect of the stimulatory agent on a corresponding control cell that lacks the regulatory element of interest or that has regulatory elements with polynucleotide sequences that are less than 60, 40, 30, 20, or 10% identical to the polynucleotide sequence of the regulatory element of interest.

Selection of Regulatory Elements that are Inhibited by a Stimulatory Agent

For the identification of regulatory elements (e.g., enhancers or promoters) that are inhibited by a stimulatory agent of interest, the cells are incubated in the presence of Zeocin to select cells containing the induction trap vector (FIG. 6). Then, the selected cells are incubated in the presence of both the stimulatory agent and gancylcovir. This incubation eliminates undesired cells containing trapped regulatory elements that are transcriptionally active in the presence of the stimulatory agent, allowing cells in which the trapped regulatory elements are inactivated by the stimulatory agent to be selected. The selected cells may be assayed to confirm that the reporter gene is transcribed in the absence of the stimulatory agent, resulting in SEAP production (FIG. 7).

Identification of Trapped Genes

The sequence of the trapped regulatory elements that are upstream of the integrated construct may be determine using standard 5′ RACE molecular biology methods, as described in Example 5. Additionally, the coding sequence for the trapped gene that is upstream and/or downstream of the integrated construct may be determined using standard DNA amplification and sequencing methods.

Alternatively, if an induction trap vector is used that contains a prokaryotic promoter (e.g., a bacterial promoter) operably linked to the positive selection marker, such as the vector illustrated in FIG. 9A, bacterial cells may be used to facilitate the identification of the trapped regulatory elements. In this method, genomic DNA from a selected eukaryotic cell is isolated and digested with a restriction enzyme that cleaves the integrated construct at one site and cleaves the endogenous, eukaryotic genomic DNA flanking the integrated construct at one or more sites. Or the DNA is digested with a restriction enzyme that does not cleave the integrated construct but cleaves the endogenous, eukaryotic genomic DNA at two or more sites. Alternatively, two restriction enzymes can be used so that one restriction enzyme cleaves the endogenous DNA and the other restriction enzyme cleaves either another site in the endogenous DNA or cleaves a site in the integrated construct.

For example, for the vector illustrated in FIG. 9A, the ClaI restriction enzyme is used to cleave the integrated construct at a single, predetermined site and to cleave the eukaryotic genomic DNA at one or more cleavage sites. The restriction enzyme-digested DNA fragments are then ligated to a restriction enzyme digested bacterial plasmid. The desired, ligated bacterial plasmids contain an insert with the positive selection marker (e.g., zeocin) from the construct that integrated into the selected eukaryotic cell and contain a region of the eukaryotic genomic DNA flanking the integrated construct. To select these desired plasmids, the plasmids are used to transform competent bacterial cells, and the transformed bacterial cells are grown on plates containing the selection agent to which the positive selection marker present within the insert confers resistance (e.g., zeocin). If a bacterial plasmid that also contains an endogenous positive selection marker is used, such as the PBSK vector that contains the ampicillin drug resistance gene, the transformed bacteria can be plated on plates containing both positive selection agents (e.g., ampicillin and zeocin). The selected bacteria contain a region from the eukaryotic genomic DNA that flanked the integrated induction trap vector. The size of this eukaryotic genomic DNA fragment can be calculated based on the size of the insert that was added to the bacterial plasmid (e.g., based on the migration in an agarose gel compared to the migration of standards with known molecular weights). The sequence of this eukaryotic genomic DNA can be readily determined by PCR amplifying and sequencing the insert in the bacterial plasmid using a primer designed to bind a region of the plasmid, such as a primer that binds the prokaryotic promoter upstream from the insert, or using a primer designed to bind a region in the insert. The sequence of the genomic DNA can be compared to known sequences, such as the publicly available sequence of the human genome, to identify the eukaryotic regulatory elements trapped by the induction trap vector and to identify the genes that are operably linked to these regulatory elements.

Similarly, if an induction trap vector is used that contains a yeast promoter operably linked to the positive selection marker, yeast cells may be used to facilitate the identification of the trapped regulatory elements. This method is performed essentially as described above, except that yeast cells are transformed with a plasmid containing a yeast promoter and an insert which includes the positive selection marker and a region of the eukaryotic, genomic DNA that flanked the integrated induction trap vector in the selected eukaryotic cells. The yeast cells containing the desired plasmid are selected using the positive selection agent, and then the insert is PCR amplified and sequenced as described above.

### EXAMPLE 10

### Identification of Genes Encoding Proteins which Modulate Regulatory Elements

As described in Example 7, genes may be identified that encode proteins which modulate the transcriptional activity of the regulatory elements identified using an induction trap vector. In one possible method, a transactivator coding sequence (e.g., teton/off) is added to the region of the induction trap vector that integrated into the genome of the isolated cells from Example 9. Any standard molecular biology technique may be used to add this transactivator coding sequence.

Cassette Exchange

For example, a vector containing an exchange cassette with the transactivator coding sequence and a reporter gene flanked by LoxP sites may be used to replace the region of the induction trap vector of Example 9 that is flanked by LoxP sites. A LoxP site consists of a double-stranded 34 basepair sequence. This sequence contains two 13 basepair inverted repeat sequences that are separated from one another by an 8 basepair spacer region (Hoess et al., Proc. Natl. Acad. Sci. U.S.A. 79:3398-3402, 1982; Sauer, U.S. Pat. No. 4,959,317). One strand of the LoxP site has the sequence 5′-ATAACTTCGTATAATGTATGCTATACGAAGTTAT-3′ (SEQ ID NO.:3), and the other strand has the sequence 5′-ATAACTTCGTATAGCATACATTATACGAAGTTAT-3′ (SEQ ID NO.:4). Alternatively, other lox sites (e.g., Lox 511 sites) or LoxP sites containing nucleotide substitutions that do not prevent recognition by the Cre recombinase may be used (Sauer, Methods: A Companion to Methods in Enzymology 14:381-392, 1998).

This Cre recombinase-mediated cassette exchange may be performed by transfecting the selected cells from Example 9 with the vector illustrated in FIG. 8B that contains the LoxP flanked exchange cassette and with a vector encoding Cre recombinase (see, for example, Fukushige and Sauer, Proc. Natl. Acad. Sci. USA 89:7905-7909, 1992; Feng et al., J. Mol. Biol. 292:779-785, 1999; U.S. Pat. No. 4,959,317; Proc. Natl. Acad. Sci. U.S.A. 85:5166-5170, 1988). Alternatively, the selected cells may be transfected with a vector that contains both the LoxP flanked exchange cassette and a Cre recombinase coding sequence. The cells in which Cre-mediated recombination has taken place may be selected based on the expression of the reporter gene from the exchange cassette (e.g., β-galactosidase) and based on Zeocin sensitivity. Expression of the transactivator polypeptide may also be confirmed by western blotting. The above method may also be used if one or both of the vectors contain only one LoxP site.

Alternatively, the cassette exchange may be performed using recombinase signal sequences and a recombinase from any other site-specific recombinase system. For example, the flp recombinase (Schwartz et al., J. Molec. Biol. 205:647-658, 1989; Parsons et al., J. Biol. Chem. 265:4527-4533, 1990; Golic et al., Cell 59:499-509, 1989; Amin et al., J. Molec. Biol. 214:55-72, 1990); the site-specific recombination system of the E. coli bacteriophage λ (Weisberg et al., In: Lambda II, (Hendrix et al., Eds.), Cold Spring Harbor Press, Cold Spring Harbor, N.Y., pp. 211-250 (1983), TpnI and the β-lactamase transposons (Levesque, J. Bacteriol. 172:3745-3757, 1990); the Tn3 resolvase (Flanagan et al., J. Molec. Biol. 206:295-304, 1989; Stark et al., Cell 58:779-790, 1989); the yeast recombinases (Matsuzaki et al., J. Bacteriol. 172:610-618, 1990); the B. subtilis SpoIVC recombinase (Sato et al., J. Bacteriol. 172:1092-1098, 1990); the Hin recombinase (Glasgow et al., J. Biol. Chem. 264:10072-10082, 1989); immunoglobulin recombinases (Malynn et al., Cell 54:453-460, 1988); or the Cin recombinase (Hafter et al., EMBO J. 7:3991-3996, 1988; Hubner et al., J. Molec. Biol. 205:493-500, 1989) can be used. These alternative systems are also discussed by Echols (J. Biol. Chem. 265:14697-14700, 1990), de Villartay (Nature 335:170-174, 1988), Craig (Ann. Rev. Genet. 22:77-105, 1988), Poyart-Salmeron et al. (EMBO J. 8:2425-2433, 1989), Hunger-Bertling et al. (Molec. Cell. Biochem. 92:107-116, 1990), and Cregg (Molec. Gen. Genet. 219:320-323, 1989).

The region of the induction trap vector that is replaced by the exchange cassette includes an IRES site; thus, replacing this region with the exchange cassette, rather than adding the exchange cassette downstream or upstream of this region, results in the elimination of this IRES site. Because multiple IRES sites near the reporter gene may decrease the transcription of the reporter gene, eliminating this IRES site may result in greater reporter gene expression than the corresponding level of reporter gene expression if this IRES site is maintained. The reporter gene in the exchange cassette (e.g., β-galactosidase) may be the same or may be different from that of the induction trap vector. Alternatively, either the induction trap vector or the exchange cassette may contain a reporter gene and the other one may lack a reporter gene. For example, if the exchange cassette does not contain a reporter gene, the integration of the exchange cassette into the genome of the cells may be determined by northern or western blotting for the encoded transactivator mRNA or protein.

The exchange cassette or the induction trap vector may optionally contain a prokaryotic or yeast promoter operably linked to a reporter gene or a positive selection marker to allow a region from the integrated construct and a region of the flanking eukaryotic, genomic DNA to be transferred to a bacterial or yeast plasmid, as described in Example 9. The bacterial or yeast plasmid can be easily produced in large quantities by the growth of bacteria or yeast transformed with the plasmid, and then PCR-amplified and sequenced to identify the trapped regulatory elements.

Introduction of Gene Trap Vector

In addition to undergoing this cassette exchange, the cells are also transfected with a gene trap vector that includes a tetracycline responsive element operably linked to a minimal promoter (e.g., TRE_{pminCMV}), a positive selective marker (e.g., Neo), an IRES sequence, and a splice donor. An exemplary construct is illustrated in FIG. 4. The gene trap vector may optionally contain a prokaryotic or yeast promoter operably linked to the positive selection marker. Transfected cells containing this construct may be selected using the positive selection drug to which the construct confers resistance. This positive selection marker may be the same or may be different from the positive selection marker in the induction trap vector encoding the transactivator.

Alternatively, a gene trap vector without a positive selection marker may be used. For example, cells containing a regulatory element that is activated by a stimulatory agent may be incubated in the absence of the stimulatory agent. Under these conditions, there is little or no expression of the positive selection marker in the induction trap vector because the stimulatory agent is not present to activate the endogenous regulatory element of interest that controls the expression of the positive selection marker. The gene trap vector is then inserted into the cells. In some or all of the cells, the TRE_{pminCMV} promoter from this vector integrates into the genome of the cells such that it is operably linked to an endogenous gene encoding a protein that activates the regulatory element of interest. The residual promoter activity of the TRE_{pminCMV} promoter in the absence of the stimulatory agent activates the transcription of the trapped gene, and then the encoded protein activates the expression of the positive selection marker. Thus, cells containing the gene trap vector may be selected based on the increased expression of the positive selection marker in the induction trap vector.

Similarly, cells containing a regulatory element that is inactivated by a stimulatory agent may be incubated in the presence of the stimulatory agent. Under these conditions, there is little or no expression of the positive selection marker in the induction trap vector because the stimulatory agent inhibits the endogenous regulatory element controlling the expression of the positive selection marker. If the TRE_{pminCMV} promoter from the gene trap vector integrates into the genome of the cells upstream of an endogenous gene encoding a protein that activates the regulatory element of interest, the encoded protein activates the expression of the positive selection marker, allowing cells containing the gene trap vector to be selected based on the increased expression of the positive selection marker.

Selection of Genes Encoding Proteins that Activate Regulatory Elements of Interest

To identify genes that activate the regulatory elements discovered in Example 9, the cells containing the exchange cassette and the gene trap vector are cultured in the presence of tetracycline, which forms a complex with the protein encoded by the teton/off nucleic acid. This complex activates expression of genes downstream of minimal promoters including tetracycline responsive elements. Thus, if the gene trap vector has integrated upstream of a gene encoding a protein that activates the regulatory element of interest, the encoded protein increases the level of transcription of the reporter gene (e.g., β-galactosidase) that is downstream of the regulatory element of interest. Culturing these cells in the presence of tetracycline leads to greater expression of the reporter gene than the corresponding level in the absence of tetracycline. These desired cells are selected based on their increased level of reporter gene expression or activity.

Selection of Genes Encoding Proteins that Inhibit Regulatory Elements of Interest

For the identification of genes encoding proteins that inactivate the regulatory elements discovered in Example 9, the cells are also cultured in the presence of tetracycline. Cells in which the gene trap vector has integrated upstream of a gene encoding a protein that inhibits the regulatory element of interest have lower levels of reporter gene expression in the presence of tetracycline than in the absence of tetracycline. Thus, these desired cells may be selected based on the inhibition of reporter gene expression or activity.

Identification of Trapped Genes

The sequence of the trapped genes that are downstream of the integrated construct may be determine using standard DNA amplification and sequencing methods. Alternatively, if the gene trap vector contains a prokaryotic or yeast promoter operably linked to a positive selection marker, bacterial or yeast cells may be used to facilitate the identification of the trapped genes as described in Example 9.

### EXAMPLE 11

### Selection of Cell Lines Responsive to Pro-Inflammatory Ligands

Cell lines were generated that are responsive to ligands in pro-inflammatory pathways involved in rheumatoid arthritis (RA), which is an auto-immune disease associated with recurrent and progressive pain and inflammation of joints. NSAID agents are commonly used to reduce the pain and signs of inflammation in rheumatoid arthritis patients. Cells involved in rheumatoid arthritis include fibroblasts and CD4⁺T cells. Members of the cytokine and chemokine signaling pathway in rheumatoid arthritis include TNFα, IL-6, IL-1β, and SDF-1.

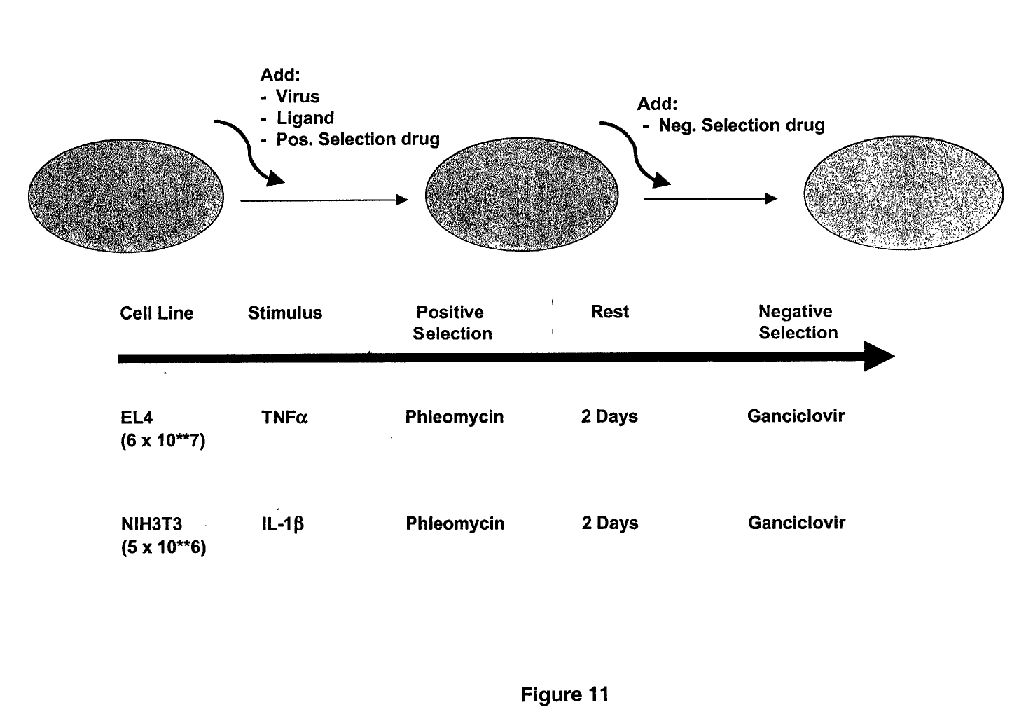

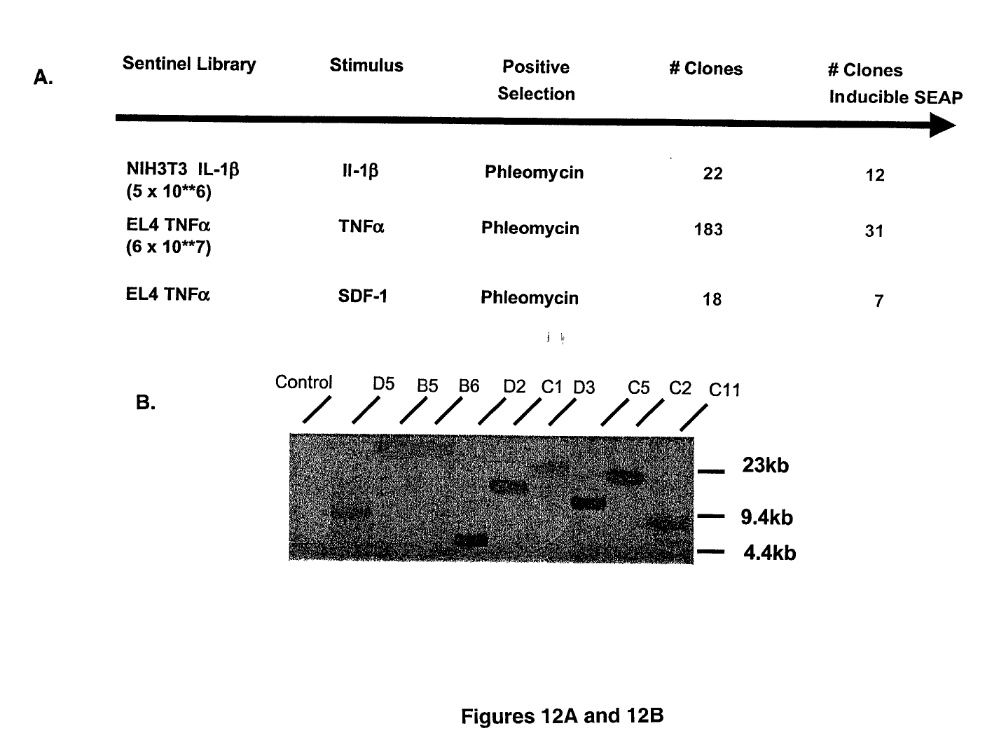

As illustrated in FIG. 11, the methods described herein were used to isolate EL4 or NIH3T3 fibroblast cells activated by TNFα or IL-1β. To confirm that the reporter gene (SEAP) that integrated into the genome of these cells was integrated under the control of a regulatory element responsive to TNFα or IL-1β, the selected cells were exposed to TNFα or IL-1β, and SEAP activity was measured (FIG. 12A). As expected, SEAP activity was induced by IL-1β in NIH3t3 cells selected for their responsiveness to IL-1β and by TNFα in EL4 cells selected for their responsiveness to TNFα. SEAP activity was also induced by SDF-1 in some of the cells selected for their responsiveness to TNFα. As illustrated in FIG. 12B, a probe to the TK/Zeo selection markers in the integrated construct was used in standard southern blot analysis to confirm the integration of the construct in some of the selected NIH3T3 cell lines.

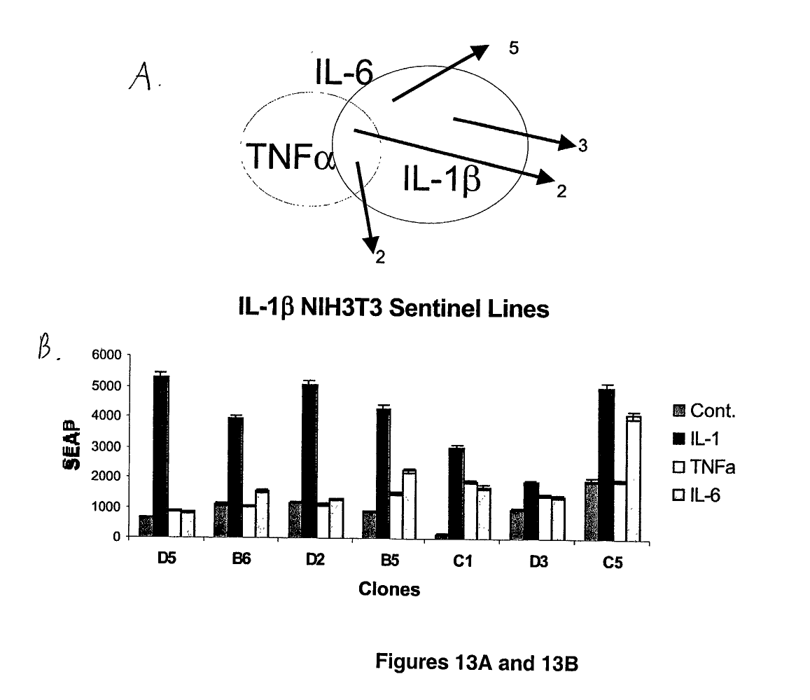

The NIH3T3 cells selected for their responsiveness to IL-1β were tested to determine whether they were also responsive to other pro-inflammatory molecules. As illustrated in FIGS. 13A and 13B, seven clones had the highest level of responsiveness to IL-1β, based on SEAP activity. The clones had varying levels of responsiveness to TNFα and IL-6. The clones that were responsive to all three ligands demonstrate that some of the pathways activated by IL-1β, TNFα, and IL-6 overlap. The clones, such as D5, that were responsive to IL-1β but had negligible response to TNFα and IL-6 demonstrate that there are IL-1β specific pathways that are independent of TNFα and IL-6.

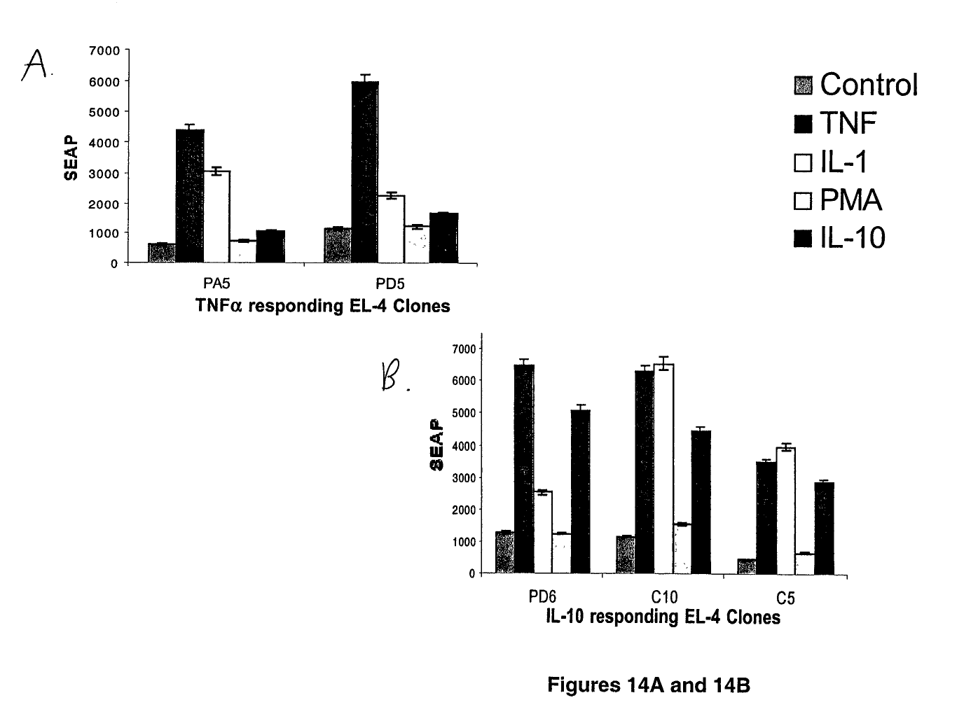

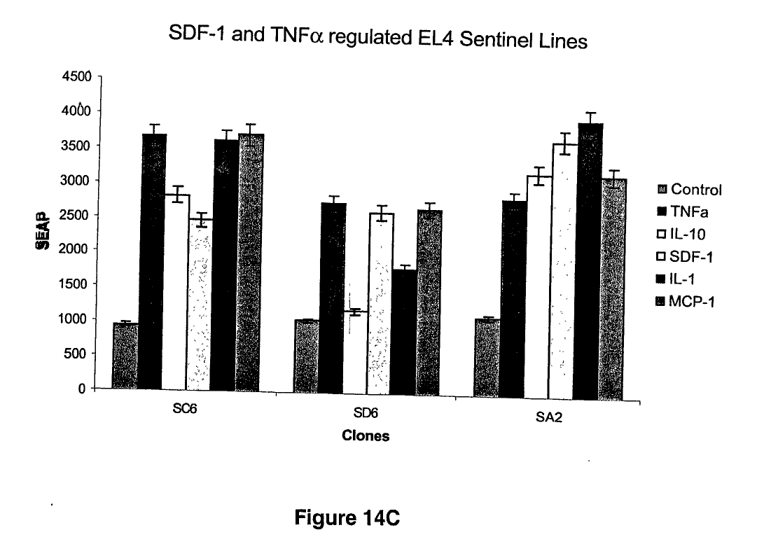

Similarly, EL-4 clones selected for their responsiveness to TNFα were tested to determine whether they were responsive to other ligands. Two clones were also responsive to IL-1, PMA, and IL-10; in order of decreasing responsiveness (FIG. 14A). EL-4 clones selected for their responsiveness to IL-10 were also responsive to TNFα and IL-1 (FIG. 14B). These results indicate that there are overlapping pathways involving TNFα, IL-1, PMA, and IL-10. EL4 cells responsive to both TNFα and SDF-1 were selected by treating TNF-α cell lines with SDF-1 in the presence of the positive selection drug (FIG. 14C).

### EXAMPLE 12

### Demonstration of a Link Between Cox-2 Activity and a Pro-Inflammatory Signaling Pathway

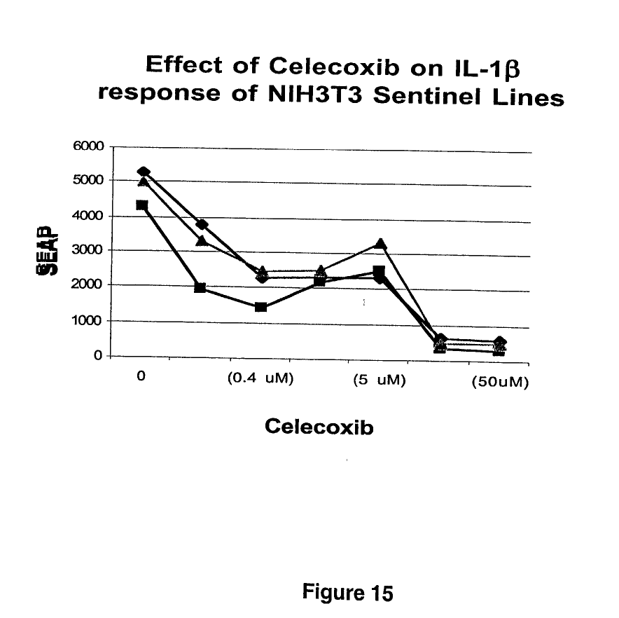

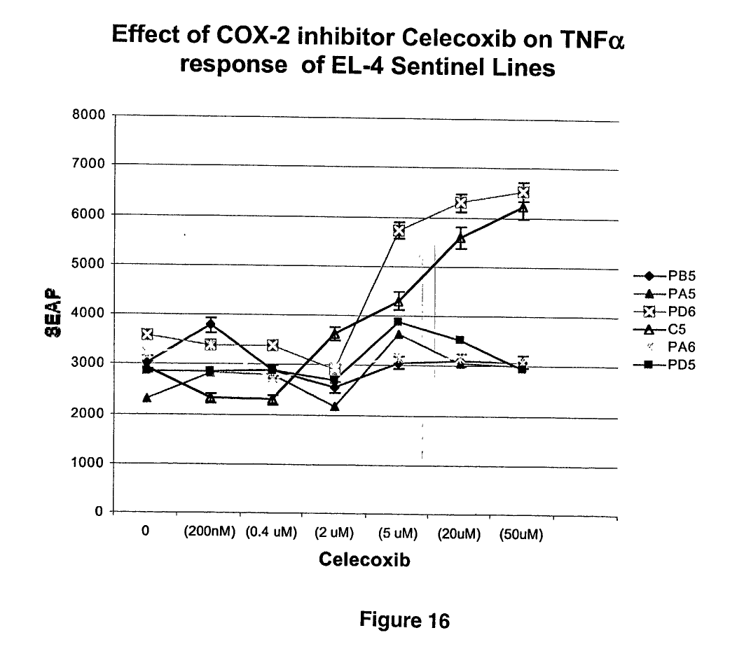

As illustrated in FIG. 15, the specific Cox-2 inhibitor, celecoxib, was shown to inhibit the effect of IL-1β on SEAP reporter gene activity in selected NIH3T3 cells in a concentration dependent manner. The IC₅₀ value of the inhibition of SEAP activity by celecoxib was approximately 0.2 uM. In contrast, celecoxib was ineffective at inhibiting the effect of TNFα on SEAP reporter gene activity in selected EL-4 cells. In some clones, TNFα increased SEAP reporter gene (FIG. 16). The clones not affected by the celecoxib include PD-5, PA-6, PA-5, and PB-5. The clones affected by celecoxib include PD-6 and C-5. Thus, within a targeted cell type, some clones are affected by the Cox-2 inhibitor and some clones are not affected.

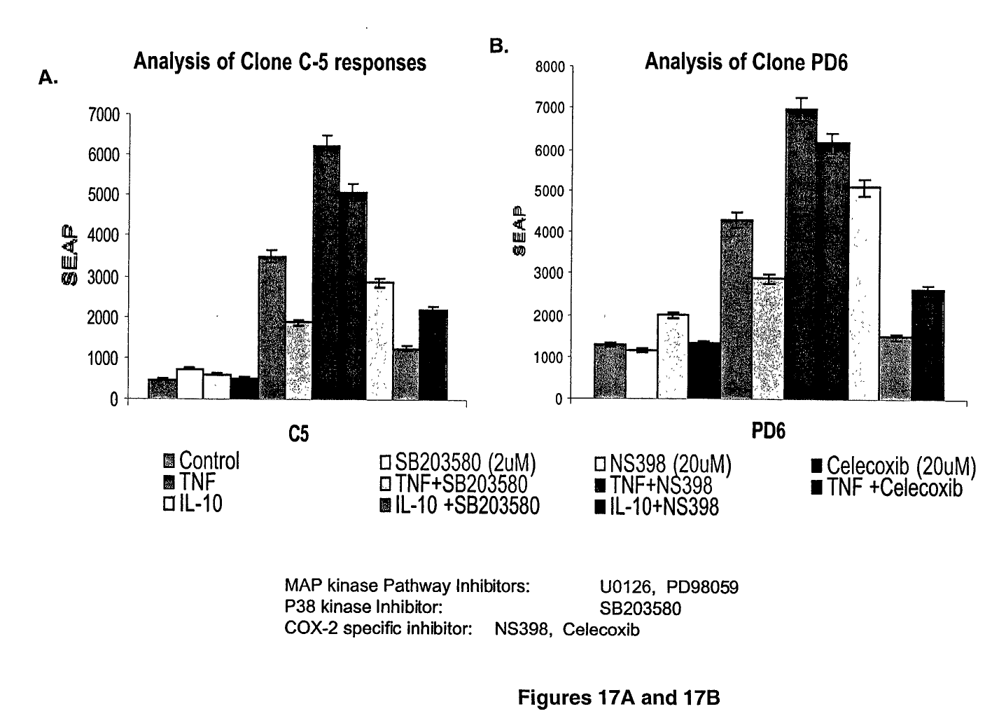

As illustrated in FIGS. 17A and 17B, different selected EL-4 clones had different levels of response to various ligands and ligand combinations. These results indicate that candidate drug products may have effects in multiple pathways. In some cases, the effect of a candidate drug in one or more pathways leads to adverse side-effects when the drug is administered to mammals (e.g., humans). Thus, candidate drugs that are identified as activating a pathway associated with adverse effects, such as toxic effects or the promotion of a disease state, are desirably eliminated from further drug development. Similarly, candidate compounds identified as inhibiting a pathway associated with beneficial effects (e.g., the reduction of adverse effects, the prevention of a disease state, or the inhibition of the progression of a disease state) are desirably eliminated from further drug development.

### EXAMPLE 13

### Use of Selected Cells to Measure Drug Efficacy

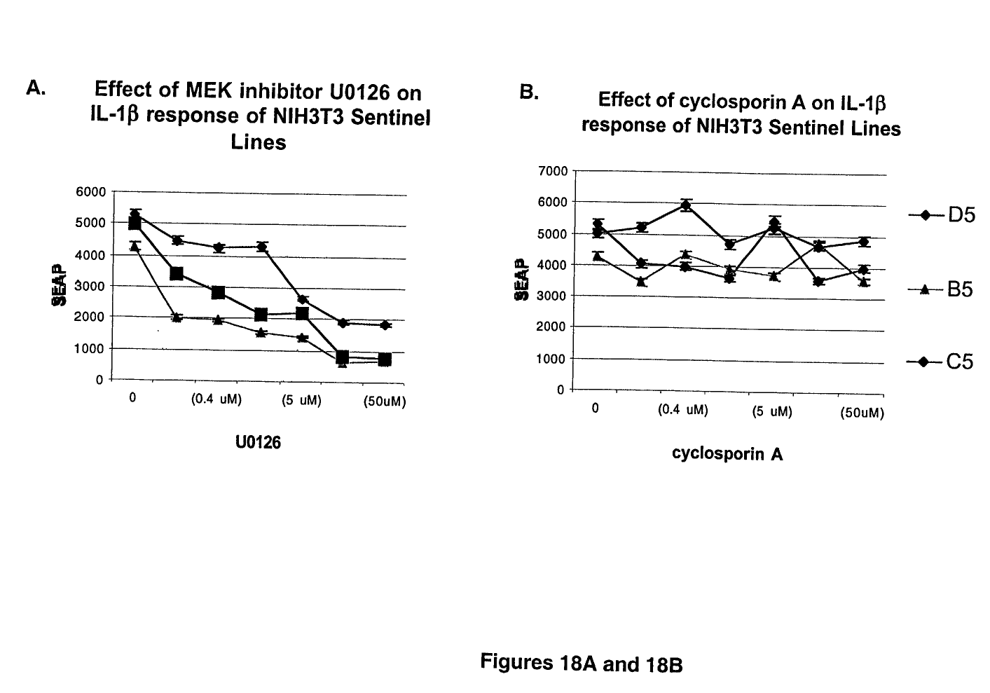

The NIH3T3 cells selected for their responsiveness to IL-1β were also tested to measure the efficacy of the MEK inhibitor U0126 and cyclosporin A. As illustrated in FIG. 18A, U0126 inhibited the effect of IL-1β on SEAP reporter gene activity in selected NIH3T3 cells in a concentration dependent manner. The IC₅₀ value of the inhibition of SEAP activity by MAP kinase inhibitors U0126 and PD98059 was approximately 1.0 uM. Cyclosporin A had a much smaller effect on SEAP activity (FIG. 18B). Thus, these selected cells are useful for measuring the activity of candidate drug products in cell-based assays.

### EXAMPLE 14

### Development of Bioassays for Mutagenic Agents

The genetic integrity of DNA is constantly being challenged by an array of DNA damaging agents, which can be either endogeneous or exogeneous in origin. Cellular repair systems are present to counteract potentially mutagenic or cytotoxic consequences from the DNA damage. Base damage is repaired either directly, through dealkylation, or via complex and coordinated pathways involving multiple proteins. These latter systems include mismatch repair (MMR), base excision repair (BER) and nucleotide excision repair. In addition, cellular regulatory pathways are activated by damaged DNA and can serve as a reporter system for the presence of mutagenic agents.

Development of cell lines that report activity of regulatory pathways activated upon exposure of cells to DNA damaging agents such as alkaylating agents enables the development of an early screen against such agents and can be used to identify compounds that cause damage to DNA.

For development of such assays a library of NIH3t3 fibroblast cell lines were generated that are responsive to the presence of the DNA-alkylating agent methyl methanesulphonate (MMS). These cells were exposed to MMS (0.2 nM) in the presence of the virus made by the viral construct as described in example 2, and the positive selection drug phelomycin. Cells surviving this selection were rested for 2 days before treatment with the negative selection marker Gancyclovir in the absence of MMS. Cell clones that demonstrated inducible SEAP reporter response upon treatment with MMS were isolated. The SEAP reporter response to other DNA mutagens were tested and clones which showed consistent response to such agents were chosen.

Genes down regulated by the presence of DNA mutagens can also be identified by reversing the order of treatment with the positive and negative selection drugs.

Identification of Trapped Genes

The sequence of the trapped regulatory elements upstream of the integrated construct may be determined using standard 5′ RACE molecular biology methods, as described in Example 5. Additionally, the coding sequence for the trapped gene (mRNA) that is upstream and/or downstream of the integrated construct may be determined using standard cDNA amplification and sequencing methods. Identification of mRNA's regulated by DNA mutagens will enable the isolation of the regulatory sequences (promoters) of these genes. Constructs utilizing promoters of these regulated genes can be made to drive expression of reporter genes. These constructs can be transfected into cells and the cells used as reporter cells for DNA damaging agents. Also, other techniques such as differential display, PCR select (Clontech) and DNA chip can be utilized to identify genes regulated by DNA mutagens. Monitoring the expression of these genes or their products, in addition to the activity of their promoters, can be used either directly or indirectly as markers for the presence of DNA damaging agents in cells.

Alternatively, if an induction trap vector is used that contains a prokaryotic promoter (e.g., a bacterial promoter) operably linked to the positive selection marker, such as the vector illustrated in FIG. 9A, bacterial cells may be used to facilitate the identification of the trapped regulatory elements. In this method, genomic DNA from a selected eukaryotic cell is isolated and digested with a restriction enzyme that cleaves the integrated construct at one site and cleaves the endogenous, eukaryotic genomic DNA flanking the integrated construct at one or more sites. Or the DNA is digested with a restriction enzyme that does not cleave the integrated construct but cleaves the endogenous, eukaryotic genomic DNA at two or more sites. Alternatively, two restriction enzymes can be used so that one restriction enzyme cleaves the endogenous DNA and the other restriction enzyme cleaves either another site in the endogenous DNA or cleaves a site in the integrated construct. These constructs would contain the regulatory sequence (promoter) of the gene regulated and these DNA can be sequenced and identified. Reporter constructs can be engineered that contain these sequences and they can be transfected into eukaryotic cells and the cells can be used as assays for the presence of DNA mutagens.

Other Embodiments

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

What is claimed is:

1. A method of selecting for one or more cells having a specific response to a stimulatory agent of interest, said method including the steps of:
(a) inserting a vector including a cassette comprising a positive selection marker, a negative selection marker, and a reporter gene into eukaryotic cells under conditions that result in the integration of said cassette into the genome of said cells, whereby said reporter gene is operably linked to a regulatory element in at least one cell; and
(b) selecting cells in which expression of said reporter gene is specifically activated by said stimulatory agent.

2. The method of claim 1, wherein step (b) comprises
(i) incubating said cells in the presence of said stimulatory agent and a positive selection agent; and
(ii) incubating said cells under conditions in which a negative selection agent is present and said stimulatory agent is absent.

3. The method of claim 1, wherein step (b) comprises
(i) incubating said cells in the presence of a positive selection agent;
(ii) incubating said cells in the presence of a negative selection agent;
(iii) incubating said cells in the presence of said stimulatory agent; and
(iv) selecting said cells that express said reporter gene in the presence of said stimulatory agent.

4. The method of claim 1, wherein said vector does not contain a promoter operably linked to said reporter gene.

5. The method of claim 1, wherein said cells are selected from the group consisting of mast cells, stem cells, epithelial cells, fibroblast cells, cancer cells, lymphocytes, and liver cells.

6. The method of claim 1, wherein said stimulatory agent is selected from the group consisting of cytokines, ligands, polypeptides, growth factors, antibodies, and chemical agents.

7. The method of claim 6, wherein said stimulatory agent is selected from the group consisting of stem cell factor, IL-3, IL-2, IL-6, IL-18, IgE, FGF-1, FGF-2, FGF-3, TGF-β, TNF-β, TNF-α, VEGF, and leptin.

8. The method of claim 1, wherein the reporter gene encodes an enzyme.

9. The method of claim 8, wherein said enzyme is selected from the group consisting of secreted alkaline phosphatase, β-galactosidase, luciferase, and green fluorescent protein.

10. The method of claim 1, wherein said vector further comprises a nucleic acid segment encoding a transactivator polypeptide, and wherein said nucleic acid is integrated into the genome of said cells.

11. The method of claim 10, wherein said transactivator polypeptide is a tetracycline regulator protein (tTA).

12. A method of selecting for one or more cells having a specific response to a stimulatory agent of interest, said method including the steps of:
(a) inserting a vector including a cassette comprising a positive selection marker, a negative selection marker, and nucleic acid segment encoding a transactivator polypeptide into eukaryotic cells under conditions that result in the integration of said cassette into the genome of said cells, whereby said nucleic acid segment encoding a transactivator polypeptide is operably linked to a regulatory element in at least one cell; and
(b) selecting cells in which expression of said transactivator polypetide is specifically activated by said stimulatory agent.

13. The method of claim 12, wherein step (b) comprises
(i) incubating said cells in the presence of said stimulatory agent and a positive selection agent; and
(ii) incubating said cells under conditions in which a negative selection agent is present and said stimulatory agent is absent.

14. The method of claim 12, wherein step (b) comprises
(i) incubating said cells in the presence of a positive selection agent;
(ii) incubating said cells in the presence of a negative selection agent;
(iii) incubating said cells in the presence of said stimulatory agent; and
(iv) selecting said cells that express said reporter gene in the presence of said stimulatory agent.

15. The method of claim 12, wherein said vector does not contain a promoter operably linked to said nucleic acid segment encoding a transactivator polypeptide.

16. A method of selecting for one or more cells having a specific response to a stimulatory agent of interest, said method including the steps of:
(a) inserting a vector including a cassette comprising a positive selection marker, a negative selection marker, and a reporter gene into eukaryotic cells under conditions that result in integration of said cassette into the genome of said cells, whereby said reporter gene is operably linked to a regulatory element in at least one cell; and
(b) selecting cells in which expression of said reporter gene is specifically inactivated by said stimulatory agent.

17. The method of claim 16, wherein step (b) comprises
(i) incubating said cells in the presence of a positive selection agent; and
(ii) incubating said cells in the presence of said stimulatory agent and a negative selection agent.

18. The method of claim 16, wherein said vector does not contain a promoter operably linked to said reporter gene.

19. The method of claim 16, wherein said cells are selected from the group consisting of mast cells, stem cells, epithelial cells, fibroblast cells, cancer cells, lymphocytes, and liver cells.

20. The method of claim 16, wherein said stimulatory agent is selected from the group consisting of cytokines, ligands, polypeptides, growth factors, antibodies, and chemical agents.

21. The method of claim 20, wherein said stimulatory agent is selected from the group consisting of stem cell factor, IL-3, IL-2, IL-6, IL-18, IgE, FGF-1, FGF-2, FGF-3, TGF-β, TNF-β, TNF-α, VEGF, and leptin.

22. The method of claim 16, wherein the reporter gene encodes an enzyme.

23. The method of claim 22, wherein said enzyme is selected from the group consisting of secreted alkaline phosphatase, β-galactosidase, luciferase, and green fluorescent protein.

24. The method of claim 16, wherein said vector further comprises a nucleic acid segment encoding a transactivator polypeptide, and wherein said nucleic acid is integrated into the genome of said cells.

25. The method of claim 24, wherein said transactivator polypeptide is tTA.

26. A method of selecting for one or more cells having a specific response to a stimulatory agent of interest, said method including the steps of:
(a) inserting a vector including a cassette comprising a positive selection marker, a negative selection marker, and a nucleic acid segment encoding a transactivator polypeptide into eukaryotic cells under conditions that result in integration of said cassette into the genome of said cells, whereby said nucleic acid segment encoding a transactivator polypeptide is operably linked to a regulatory element in at least one cell; and
(b) selecting cells in which expression of said transactivator polypeptide is specifically inactivated by said stimulatory agent.

27. The method of claim 26, wherein step (b) comprises
(i) incubating said cells in the presence of a positive selection agent; and
(ii) incubating said cells in the presence of said stimulatory agent and a negative selection agent.

28. The method of claim 26, wherein said vector does not contain a promoter operably linked to said nucleic acid segment encoding a transactivator polypeptide.

29. The method of claim 1, 12, 16, or 26, further comprising the step of (c) identifying said regulatory element.

30. The method of claim 29, wherein said positive selection marker is operably linked to a prokaryotic promoter in said cassette, and wherein step (c) comprises
(i) inserting a nucleic acid comprising said positive selection marker and comprising a segment of the genome flanking said cassette into bacterial cells under conditions that allow the selection of said bacterial cells expressing said positive selection marker under the control of said prokaryotic promoter;
(ii) amplifying said segment flanking said cassette; and
(iii) sequencing said amplified segment.

31. The method of claim 29, wherein said positive selection marker is operably linked to a yeast promoter in said cassette, and wherein step (c) comprises
(i) inserting a nucleic acid comprising said positive selection marker and comprising a segment of the genome flanking said cassette into yeast cells under conditions that allow the selection of said yeast cells expressing said positive selection marker under the control of said yeast promoter;
(ii) amplifying said segment flanking said cassette; and
(iii) sequencing said amplified segment.

32. A method for identifying a nucleic acid of interest that encodes a protein that modulates the activity of a regulatory element in a cell, said method including the steps of:
(a) inserting a first vector including a first cassette comprising a first positive selection marker, a negative selection marker, a reporter gene, and a nucleic acid segment encoding a transactivator polypeptide into eukaryotic cells under conditions that result in integration of said first cassette into the genome of said cells; wherein said reporter gene is operably linked to a regulatory element in at least one cell;
(b) inserting a second vector including a second cassette comprising a promoter operably linked to a responsive element that is responsive to said transactivator polypeptide into said cells under conditions that result in integration of said second cassette into the genome of said cells; wherein said promoter is operably linked to a nucleic acid of interest encoding a protein in at least one cell; and wherein said encoded protein modulates the activity of said regulatory element;
(c) selecting cells that have an altered level of reporter gene expression under conditions that activate said transactivator polypeptide; and
(d) identifying said nucleic acid of interest in at least one selected cell.

33. The method of claim 32, wherein said second vector further comprises a second positive selection marker, and wherein said second positive selection marker is integrated into the genome of said cells.

34. The method of claim 33, wherein said second positive selection marker is operably linked to a prokaryotic promoter in said second cassette, and wherein step (d) comprises
(i) inserting a nucleic acid comprising said second positive selection marker and comprising a segment of the genome flanking said second cassette into bacterial cells under conditions that allow the selection of said bacterial cells expressing said second positive selection marker under the control of said prokaryotic promoter;
(ii) amplifying said segment flanking said second cassette; and
(iii) sequencing said amplified segment.

35. The method of claim 33, wherein said second positive selection marker is operably linked to a yeast promoter in said second cassette, and wherein step (d) comprises
(i) inserting a nucleic acid comprising said second positive selection marker and comprising a segment of the genome flanking said second cassette into yeast cells under conditions that allow the selection of said yeast cells expressing said second positive selection marker under the control of said yeast promoter;
(ii) amplifying said segment flanking said second cassette; and
(iii) sequencing said amplified segment.

36. The method of claim 32, wherein said transactivator polypeptide is tTA and said responsive element comprises a tetracycline responsive element.

37. A method for identifying a nucleic acid of interest that encodes a protein that modulates the activity of a regulatory element in a cell, said method including the steps of:
(a) inserting a first vector including a first cassette comprising a positive selection marker, a negative selection marker, and a recombinase signal sequence into eukaryotic cells under conditions that result in integration of said first cassette into the genome of said cells;
(b) inserting a second vector including a second cassette that includes a recombinase signal sequence, a nucleic acid segment encoding a transactivator polypeptide, and a reporter gene into said cells under conditions that result in recombination between said recombinase signal sequence in said second vector and said recombinase signal sequence integrated into the genome of said cells such that said second cassette is integrated into the genome of at least one cell; and wherein said reporter gene is operably linked to a regulatory element in at least one cell;
(c) inserting a third vector including a third cassette comprising a promoter operably linked to a responsive element that is responsive to said transactivator polypeptide into said cells under conditions that result in integration of said third cassette into the genome of said cells; wherein said promoter is operably linked to a nucleic acid of interest encoding a protein that modulates the activity of said regulatory element in at least one cell;
(d) selecting cells that have an altered level of reporter gene expression under conditions that activate said transactivator polypeptide; and
(e) identifying said nucleic acid of interest in at least one selected cell.

38. The method of claim 37, wherein said third vector further comprises a second positive selection marker, and wherein said second positive selection marker is integrated into the genome of said cells.

39. The method of claim 38, wherein said second positive selection marker is operably linked to a prokaryotic promoter in said third cassette, and wherein step (e) comprises
(i) inserting a nucleic acid comprising said second positive selection marker and comprising a segment of the genome flanking said third cassette into bacterial cells under conditions that allow the selection of said bacterial cells expressing said second positive selection marker under the control of said prokaryotic promoter;
(ii) amplifying said segment flanking said third cassette; and
(iii) sequencing said amplified segment.

40. The method of claim 39, wherein said second positive selection marker is operably linked to a yeast promoter in said third cassette, and wherein step (e) comprises
(i) inserting a nucleic acid comprising said second positive selection marker and comprising a segment of the genome flanking said third cassette into yeast cells under conditions that allow the selection of said yeast cells expressing said second positive selection marker under the control of said yeast promoter;
(ii) amplifying said segment flanking said third cassette; and
(iii) sequencing said amplified segment.

41. The method of claim 37, wherein said transactivator polypeptide is tTA and said responsive element comprises a tetracycline responsive element.

42. The method of claim 37, wherein said recombinase signal sequence is a LoxP site.

43. The method of claim 42, wherein said first vector and/or said second vector include two LoxP sites.

44. A method for identifying a nucleic acid of interest that encodes a protein that modulates the activity of a regulatory element in a cell, said method including the steps of:
(a) inserting a first vector including a first cassette comprising a positive selection marker, a negative selection marker, a reporter gene, and a recombinase signal sequence into eukaryotic cells under conditions that result in integration of said first cassette into the genome of said cells;
(b) inserting a second vector including a second cassette that includes a recombinase signal sequence and a nucleic acid segment encoding a transactivator polypeptide into said cells under conditions that result in recombination between said recombinase signal sequence in said second vector and said recombinase signal sequence integrated into the genome of said cells such that said second cassette is integrated into the genome of at least one cell; and wherein said reporter gene is operably linked to a regulatory element in at least one cell;
(c) inserting a third vector including a third cassette comprising a promoter operably linked to a responsive element that is responsive to said transactivator polypeptide into said cells under conditions that result in integration of said third cassette into the genome of said cells; wherein said promoter is operably linked to a nucleic acid of interest encoding a protein that modulates the activity of said regulatory element in at least one cell;
(d) selecting cells that have an altered level of reporter gene expression under conditions that activate said transactivator polypeptide; and
(e) identifying said nucleic acid of interest in at least one selected cell.

45. The method of claim 44, wherein said third vector further comprises a second positive selection marker, and wherein said second positive selection marker is integrated into the genome of said cells.

46. The method of claim 45, wherein said second positive selection marker is operably linked to a prokaryotic promoter in said third cassette, and wherein step (e) comprises
(i) inserting a nucleic acid comprising said second positive selection marker and comprising a segment of the genome flanking said third cassette into bacterial cells under conditions that allow the selection of said bacterial cells expressing said second positive selection marker under the control of said prokaryotic promoter;
(ii) amplifying said segment flanking said third cassette; and
(iii) sequencing said amplified segment.

47. The method of claim 45, wherein said second positive selection marker is operably linked to a yeast promoter in said third cassette, and wherein step (e) comprises
(i) inserting a nucleic acid comprising said second positive selection marker and comprising a segment of the genome flanking said third cassette into yeast cells under conditions that allow the selection of said yeast cells expressing said second positive selection marker under the control of said yeast promoter;
(ii) amplifying said segment flanking said third cassette; and
(iii) sequencing said amplified segment.

48. The method of claim 44, wherein said transactivator polypeptide is tTA and said responsive element comprises a tetracycline responsive element.

49. The method of claim 44, wherein said recombinase signal sequence is a LoxP site.

50. The method of claim 49, wherein said first vector and/or said second vector include two LoxP sites.

51. A method for treating, preventing, or stabilizing a disease associated with a stimulatory agent, said method including the steps of:
(a) inserting a vector including a cassette comprising a positive selection marker, a negative selection marker, and a reporter gene into eukaryotic cells under conditions that result in the integration of said cassette into the genome of said cells, whereby said reporter gene is operably linked to a regulatory element in at least one cell; and
(b) selecting cells in which expression of said reporter gene is specifically modulated by said stimulatory agent;
(c) selecting a compound that increases or decreases the effect of said stimulatory agent on the expression of said reporter gene; and
(d) administering said compound to a mammal having a disease associated with said stimulatory agent.

52. A method for treating, preventing, or stabilizing a disease associated with a stimulatory agent, said method including the steps of:
(a) inserting a vector including a cassette comprising a positive selection marker, a negative selection marker, and a nucleic acid segment encoding a transactivator polypeptide into eukaryotic cells under conditions that result in the integration of said cassette into the genome of said cells, whereby said nucleic acid segment encoding a transactivator polypeptide is operably linked to a regulatory element in at least one cell; and
(b) selecting cells in which expression of said transactivator polypeptide is specifically modulated by said stimulatory agent;
(c) selecting a compound that increases or decreases the effect of said stimulatory agent on the expression of said transactivator polypeptide; and
(d) administering said compound to a mammal having a disease associated with said stimulatory agent.

53. A nucleic acid including a positive selection marker, a negative selection marker, and a reporter gene.

54. The nucleic acid of claim 53, including, in 5′ to 3′ sequence,
(a) a splice acceptor;
(b) a cassette including, in any order, a negative selection marker, and a positive selection marker;
(c) a translation stop sequence,
(d) an internal ribosome entry site, and
(e) a reporter gene.

55. The nucleic acid of claim 53 including, in 5′ to 3′ sequence,
(a) a splice acceptor;
(b) a cassette including, in any order, a negative selection marker and a reporter gene;
(c) a translation stop sequence,
(d) a promoter,
(e) a positive selection marker;
(f) a translation stop sequence; and
(g) a polyadenylation signal.

56. The nucleic acid of claim 53, wherein said reporter gene is not operably linked to a promoter in said nucleic acid.

57. The nucleic acid of claim 53, further including a nucleic acid segment encoding a transactivator polypeptide.

58. The nucleic acid of claim 53, further including one or more recombinase signal sequences.

59. The nucleic acid of claim 53, further including a prokaryotic promoter operably linked to said positive selection marker.

60. A nucleic acid including a positive selection marker, a negative selection marker, and a nucleic acid segment encoding a transactivator polypeptide.

61. A nucleic acid including a positive selection marker, a negative selection marker, and a recombinase signal sequence.

62. A nucleic acid including a splice acceptor site and a prokaryotic promoter operably linked to a positive selection marker.

63. A vector that includes the nucleic acid of claim 53, 60, 61, or 62.

64. The vector of claim 63, which is a retroviral vector.

65. The vector of claim 63, further including an integration sequence.

66. A cell including the vector of claim 63.

67. The cell of claim 66, responsive to one or more stimulatory agents.

68. A cell including (i) a first nucleic acid which includes a positive selection marker, a negative selection marker, and a nucleic acid segment encoding a transactivator polypeptide and (ii) a second nucleic acid which includes a promoter operably linked to a responsive element that is responsive to said transactivator polypeptide.

69. A screening method for selecting candidate compounds that modulate the activity of a stimulatory agent of interest, said method including the steps of:
(a) contacting one or more cells of claim 66 or 68 having a specific response to said stimulatory agent with one or more candidate compounds and said stimulatory agent; and
(b) selecting the candidate compounds which modulate said response to said stimulatory agent.

70. The method of claim 69, wherein said candidate compound increases said response to said stimulatory agent.

71. The method of claim 69, wherein said candidate compound decreases said response to said stimulatory agent.

72. A method for determining whether a candidate compound modulates the activity of a regulatory element of interest, said method including the steps of:
(a) contacting one or more cells of claim 66 or 68 having said regulatory element of interest operably linked to a positive selection marker, reporter gene, or nucleic acid segment encoding a transactivator polypeptide with one or more candidate compounds; and
(b) selecting a candidate compound which modulates the expression of said positive selection marker, reporter gene, or nucleic acid segment encoding a transactivator polypeptide, thereby selecting a candidate compound which modulates the activity of said regulatory element of interest.

73. The method of claim 72, wherein said candidate compound is eliminated from drug development.

74. The method of claim 72, wherein said method is performed prior to an animal model study or human clinical trial of said candidate compound.

75. A method for determining whether a test compound damages DNA of eukaryotic cells, said method comprising the steps of:
(a) providing a eukaryotic test cell containing regulatory DNA operatively associated with a reporter gene, wherein said regulatory DNA is derived from a gene that is activated in a cell upon damage to DNA in said cell,
(b) contacting said test compound with said test cell, and
(c) detecting said reporter as an indictor that said test compound damages DNA.
